(19) United States
(12) Patent Application Publication
(10) Pub. No.: US 2025/0179131 A1
(43) Pub. Date: Jun. 5, 2025

(54) IL-18 BINDING PROTEIN (IL-18BP) IN THE TREATMENT OF VEXAS

(71) Applicant: AB2 Bio SA, Lausanne (CH)

(72) Inventors: Djordje Filipovic, Lausanne (CH); Eduardo Schiffrin, Lausanne (CH); Cyril Portmann, Lausanne (CH); Michael Soldan, Le Mont-Pèlerin (CH); Laurence Goffin, Ornex (FR)

(21) Appl. No.: 18/843,964

(22) PCT Filed: Mar. 3, 2023
(86) PCT No.: PCT/EP2023/055498

§ 371 Date: Sep. 4, 2024

(30) Foreign Application Priority Data
Mar. 4, 2022 (EP) 22160185.9

(51) Int. Cl. C07K 14/47; A61K 38/00; A61P 29/00; G01N 33/68

(52) U.S. Cl. CPC C07K 14/4703; A61P 29/00; G01N 33/6869; G01N 33/6893; A61K 38/00; G01N 2333/4703; G01N 2333/54; G01N 2800/52

(57) ABSTRACT

The present invention provides an IL-18 inhibitor for use in the treatment of VEXAS syndrome or symptoms associated with VEXAS syndrome in a subject.

### CROSS-REFERENCE TO RELATED APPLICATIONS

This application is a section 371 of International Application No. PCT/EP2023/055498 filed Mar. 3, 2023, which was published in the English language on Sep. 7, 2023, under International Publication No. WO 2023/166206 A1, which claims priority to European Patent Application No. 22160185.9, filed on Mar. 4, 2022. Each disclosure is incorporated herein by reference in its entirety.

### REFERENCE TO SEQUENCE LISTING SUBMITTED ELECTRONICALLY

This application contains a sequence listing, which is submitted electronically. The contents of the electronic sequence listing (069069.6US1 Sequence Listing.xml; size: 7,951 bytes; and a creation date of Sep. 4, 2024) is herein incorporated by reference in its entirety.

### BACKGROUND AND SUMMARY

The present invention provides an IL-18 inhibitor for use in the treatment of VEXAS syndrome or symptoms associated with VEXAS syndrome in a subject.

VEXAS syndrome (vacuoles. E1 enzyme, X-linked, autoinflammatory, somatic) is monogenic, adult-onset autoinflammatory disease affecting mostly males, caused by a mutation in the UBA1 gene in hematopoietic progenitor cells; see Grayson et al. (2021) Blood 137 (26): 3591-4. The syndrome was first reported by Beck et al, who write: “Using a genotype-driven approach, we identified a disorder that connects seemingly unrelated adult-onset inflammatory syndromes.”; see Beck D B, Ferrada M A, Sikora K A, et al. (2020) N. Engl. J. Med. 383 (27): 2628-2638. Myeloid cells driven autoinflammatory reactions and progressive bone marrow dysfunction are the central characteristics of the disease.

This newly identified disorder was now surprisingly correlated with an increased level of cytokines, in particular IL-18. It was thus envisaged by the present inventors that a therapeutic approach using IL-18BP may be employed, optionally combined with a quantification of free IL-18 in the body fluids and/or body tissues of the patient to be treated by using a quantification assay as described in WO 2015/032932 and WO 2016/139297, respectively, the disclosures of which are incorporated herein by reference. Bourbon et al. (2021) Blood 137, No. 26 describes potential treatment options in VEXAS syndrome. Kirino et al. (2021) Annals of the Rheumatic Diseases 80, no. 11, describes the use of tocilizumab in VEXAS, an IL-6 receptor antibody. Lastly, Wiesik-Szewczyk (2021) Reumatologica 59, no. 6, describes IL-1 inhibitors and their uses.

Accordingly, the present invention solves the problem of a lack of effective treatment options for VEXAS patients.

That is, the present invention relates to, inter alia, the following embodiments:
1. An IL-18 inhibitor for use in the treatment of VEXAS syndrome or symptoms associated with VEXAS syndrome in a subject.
2. The IL-18 inhibitor for use of embodiment 1, wherein symptoms associated with VEXAS syndrome are characterized by autoinflammatory manifestations, in particular severe autoinflammatory manifestations, and/or hyperinflammation, in particular hyperinflammation as characterized by known inflammation markers, in particular elevated known inflammation markers, such as CRP.
3. The IL-18 inhibitor for use of embodiment 1, wherein the subject has one or more mutations in the UBA1 gene, in particular at gene locus p11.3 on the X-chromosome.
4. The IL-18 inhibitor for use of embodiment 3, wherein the mutation(s) result(s) in an alternative, in particular shorter, isoform of the UBA-1 gene product.
5. The IL-18 inhibitor for use of embodiment 4, wherein the mutation(s) result(s) in a M41T, M41V or M41L substitution.
6. The IL-18 inhibitor for use of any one of embodiments 1 to 5, wherein treatment is achieved and/or supported by blocking the proinflammatory activity of IL-18.
7. The IL-18 inhibitor for use of any one of embodiments 1 to 6, which is an IL-18 binding protein (IL-18BP), including any functional equivalent or functional part thereof which retains the capability of blocking the proinflammatory activity of IL-18.
8. The IL-18 inhibitor for use of embodiment 7, which is a human IL-18BP (hIL-18 BP), including any functional equivalent or functional part thereof which retains blocking of the proinflammatory activity of IL-18.
9. The IL-18 inhibitor for use of embodiment 8, which is a recombinant human IL-18BP (rhIL-18 BP), including any functional equivalent or functional part thereof which retains the capability of blocking the proinflammatory activity of IL-18.
10. The IL-18 inhibitor for use of embodiment 8 or embodiment 9, wherein said human IL-18BP is selected from isoform a, b, c and d of human IL-18BP, particularly isoform a as in SEQ ID NO: 2, isoform b as in SEQ ID NO: 3, isoform c as in SEQ ID NO: 4 or isoform d as in SEQ ID NO: 5, including any functional equivalent or functional part of isoforms a, b, c and/or d which retains the capability of blocking the proinflammatory activity of IL-18.
11. The IL-18 inhibitor for use of embodiment 8 or embodiment 9, which is an IL-18BP as shown in SEQ ID NO: 2, including any functional equivalent or functional part thereof which retains the capability of blocking the proinflammatory activity of IL-18.
12. The IL-18 inhibitor for use of embodiment 11 wherein the functional equivalent has a sequence identity of 80%, 85%, 90%, 91%, 92%, 93%, 94%, 95%, 96%, 97%, 98%, 99% to the sequence depicted in SEQ ID NO: 2 and retains the capability of blocking the proinflammatory activity of IL-18.
13. The IL-18 inhibitor for use of any one of embodiments 7 to 12, wherein the functional equivalent or functional part thereof includes a mutein of IL-18BP, a fragment, a peptide, a functional derivative, a functional fragment, a fraction, a circularly permuted derivative, a fused protein comprising IL-18BP, an isoform or a salt thereof which retains the capability of blocking the proinflammatory activity of IL-18.
14. The IL-18 inhibitor for use of any one of embodiments 7 to 13 comprising in addition to the IL-18 binding protein (IL-18BP), N-terminal and/or C-terminal deletion variants of IL-18BP in an amount of up to 40%, particularly up to 30%, particularly up to 20%, particularly up to 15%, particularly up to 10%, particularly up to 7.5%, particularly up to 5%, particularly up to 2.5%, particularly up to 1%, particularly up to 0.5%, particularly up to 0.25%, particularly up to 0.1%, particularly up to 0.05%, particularly up to 0.01%.
15. The IL-18 inhibitor for use according to embodiment 14, wherein said deletion variants comprise deletions of between 1 and 5 amino acid residues at the C-terminal end of the IL-18BP and/or between 1 and 30 amino acid residues at the N-terminal end of the IL-18BP.
16. The IL-18 inhibitor for use according to embodiment 14 or embodiment 15, wherein said N-terminal and/or C-terminal deletion variants of IL-18BP are present in an amount of up to 40%, particularly in an amount of between 2% and 35%.
17. The IL-18 inhibitor for use of any one of embodiments 1 to 16, wherein body fluids and/or body tissues of the subject to be treated have been quantified to have abnormal levels of free IL-18, which exceed the level of free IL-18 in body fluids and/or body tissues of a healthy control subject, particularly by 5%, 10%, 20%, 30%, 40%, 50%, 60%, 70%, 80%, 90%, 100%, or more than 100%, using an assay capable of detecting free IL-18 in body fluids and/or body tissues.
18. The IL-18 inhibitor for use of embodiment 17, wherein the subject to be treated has a level of free IL-18 above about 2.7 pg/ml, in particular above about 8 pg/ml or wherein the body fluids and/or body tissues of the subject to be treated have been quantified to have a level of free IL-18 above about 2.7 pg/ml, in particular above about 8 pg/ml.
19. The IL-18 inhibitor for use of any one of embodiments 1 to 18, wherein the subject has an abnormal, elevated level of total IL-18.
20. The IL-18 inhibitor for use of embodiment 19, wherein the IFN-γ-mediated IL-18BP induction is impaired.
21. The IL-18 inhibitor for use of any one of embodiments 17 to 20, wherein the level of total IL-18 is above about 250 pg/ml, in particular above about 1000 pg/ml, more particular above about 3000 pg/ml or wherein the body fluids and/or body tissues of the subject to be treated have been quantified to have a level of total IL-18 above about 250 pg/ml, in particular above about 1000 pg/ml, more particular above about 3000 pg/ml.
22. The IL-18 inhibitor for use of any one of embodiments 17 to 21, wherein the subject has a level of ferritin above 400 ng/ml, preferably above 1000 ng/ml or wherein the body fluids and/or body tissues of the subject to be treated have been quantified to have a level of ferritin above 400 ng/ml, preferably above 1000 ng/ml.
23. The IL-18 inhibitor for use to any one of embodiments 17 to 22, wherein quantifying the level of free IL-18 in the body fluids and/or body tissues comprises the following steps:
   a) bringing a sample of body fluid and/or body tissue suspected to contain free IL-18 into contact with the IL-18 inhibitor as defined in any one of embodiments 7 to 16 as the capturing molecule for free IL-18;
   b) allowing the IL-18 inhibitor to bind free IL-18;
   c) detecting the binding of the IL-18 inhibitor and determining the amount of free IL-18 in the sample.
24. The IL-18 inhibitor for use of any one of embodiments 17 to 23, wherein the body fluids and/or body tissues are selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa, bone-marrow or tissue secretion.
25. A composition for use in the treatment of VEXAS syndrome or symptoms associated therewith comprising an IL-18 inhibitor as defined in any one of embodiments 7 to 16 and a pharmaceutically acceptable carrier and/or excipient.
26. The IL-18 inhibitor for use of any one of embodiments 1 to 24 or the composition for use of embodiment 25, wherein said IL-18 inhibitor or composition is administered to a subject in need thereof in a single dose/day, in multiple doses/day, in multiple doses/week or in multiple doses/month.
27. The IL-18 inhibitor for use of any one of embodiments 1 to 24 or the composition for use of embodiment 25 or embodiment 26, wherein said IL-18 inhibitor or composition is administered in one dose per week, in two doses per week, three doses per week, four doses per week, five doses per week, six doses per week, but particularly seven doses per week, preferably three or four doses per week.
28. The IL-18 inhibitor for use of any one of embodiments 1 to 24 or the composition for use of embodiment 25 or embodiment 26, wherein said IL-18 inhibitor or composition is administered every 24 h to 48 h, preferably every 48 h.
29. The IL-18 inhibitor for use of any one of embodiments 1 to 24 or the composition for use of embodiment 25 or embodiment 26, wherein said IL-18 inhibitor or composition is administered in a single dose every other day, for example over 3 weeks.
30. The IL-18 inhibitor for use of any one of embodiments 1 to 24 and 26 to 29 or the composition for use of any one of embodiments 25 to 29, wherein a single dose comprises between 0.5 mg of IL-18 inhibitor/kg body weight and 10 mg IL-18 inhibitor/kg body weight, particularly between 1 mg IL-18 inhibitor/kg body weight and 8 mg IL-18 inhibitor/kg body weight, particularly between 1.5 mg IL-18 inhibitor/kg body weight and 6 mg IL-18 inhibitor/kg body weight, particularly between 2 mg IL-18 inhibitor/kg body weight and 4 mg IL-18 inhibitor/kg body weight.
31. The IL-18 inhibitor for use of any one of embodiments 1 to 24 and 26 to 30 or the composition for use of any one of embodiments 24 to 30, wherein a single dose of between 0.5 mg IL-18 inhibitor/kg body weight and 5 mg IL-18 inhibitor/kg body weight is administered every 24 or 48 h, particularly, wherein a single dose of 2 mg IL-18 inhibitor/kg body weight is administered every 48 h.
32. The IL-18 inhibitor for use of any one of embodiments 1 to 24 and 26 to 31 or the composition for use of any one of embodiments 25 to 31, wherein the subject to be treated is a mammal.
33. The IL-18 inhibitor for use or the composition for use of embodiment 32, wherein the subject to be treated is a human.
34. A recombinant human IL-18BP (rhIL-18 BP) or a composition comprising the recombinant human IL-18BP (rhIL-18 BP), including any functional equivalent or functional part thereof which retains the capability of blocking the proinflammatory activity of IL-18, for use in the treatment of VEXAS syndrome or symptoms associated therewith in a human, preferably wherein the human has a detectable level of free IL-18, particularly wherein said recombinant human IL-18BP (rhIL-18 BP) or the composition comprising the recombinant human IL-18BP (rhIL-18 BP) is administered to said human in a single dose every 48 h of 2 mg/kg body weight.
36. The recombinant human IL-18BP or the composition comprising the recombinant human IL-18BP (rhIL-18 BP), including any functional equivalent or functional part thereof for use according to embodiment 35, wherein the human patient suffering from VEXAS syndrome or symptoms associated therewith shows uncontrolled systemic inflammatory reactions and has abnormal, elevated levels of total IL-18.
37. The recombinant human IL-18BP or the composition comprising the recombinant human IL-18BP (rhIL-18 BP), including any functional equivalent or functional part thereof for use according to embodiment 36, wherein the level of free IL-18 in the body fluids or body tissues of the human patient is above 2.7 pg/mL.
38. The recombinant human IL-18BP or the composition comprising the recombinant human IL-18BP (rhIL-18 BP), including any functional equivalent or functional part thereof for use according to embodiment 37, wherein the level of total IL-18 is above about 250 pg/ml, in particular above about 1000 pg/ml, more particular above about 3000 pg/ml or wherein the body fluids and/or body tissues of the subject to be treated have been quantified to have a level of total IL-18 above about 250 pg/ml, in particular above about 1000 pg/ml, more particular above about 3000 pg/ml.
39. A recombinant human IL-18BP (rhIL-18 BP) or a composition comprising the recombinant human IL-18BP (rhIL-18 BP), including any functional equivalent or functional part thereof which retains the capability of blocking the proinflammatory activity of IL-18, for use in the treatment of VEXAS syndrome or associated symptoms therewith in a human, optionally a human with confirmed VEXAS syndrome and, wherein the human has a level of ferritin above 400 ng/ml, preferably above 1000 ng/ml.
40. The recombinant human IL-18BP or the composition comprising the recombinant human IL-18BP (rhIL-18 BP), including any functional equivalent or functional part thereof for use according to embodiment 39, wherein the level of total IL-18 is above about 250 pg/ml, in particular above about 1000 pg/ml, more particular above about 3000 pg/ml or wherein the body fluids and/or body tissues of the subject to be treated have been quantified to have a level of total IL-18 above about 250 pg/ml, in particular above about 1000 pg/ml, more particular above about 3000 pg/ml.

Accordingly, in its broadest aspect, the present invention relates to an IL-18 inhibitor for use in the treatment of VEXAS syndrome or symptoms associated with VEXAS syndrome in a subject. Herein, the treatment of VEXAS syndrome per se is a preferred embodiment of the invention. However, it is also provided herein to treat, prevent and/or alleviate symptoms associated with VEXAS, in particular via the treatment of VEXAS per se.

The clinical manifestations of VEXAS syndrome may present different phenotypes, amongst which are fever, fatigue, weight loss, lymphadenopathies, arthralgia/arthritis, skin nodular lesions as frequently observed manifestations/symptoms. Further frequent findings in laboratory tests comprise anemia, neutropenia, thrombocytopenia, and/or an increase in blood markers such as CRP and ferritin. Myeloid cell vacuolization is a characteristic sign of VEXAS. Hemophagocytosis is frequently observed in bone marrow that can show, in addition, signs of myelofibrosis. Recurrent skin rash and symmetric polyarthritis are also known as manifestations/symptoms. Inflammation markers are usually elevated, at least during an active phase of the disease, i.e. a phase where symptoms are observed (Staels, F., et al. (2021). Frontiers in Immunology 12). Accordingly, the present invention, in some embodiments, relates to an IL-18 inhibitor for use in the alleviation of symptoms associated with VEXAS syndrome, preferably in a patient diagnosed with VEXAS syndrome, in particular one or more of fever, fatigue, weight loss, lymphadenopathies, arthralgia/arthritis, skin nodular lesions.

### DETAILED DESCRIPTION

Within the present invention, treatment of VEXAS syndrome of symptoms associated with VEXAS syndrome is preferably achieved and/or supported by blocking the proinflammatory activity of IL-18.

Accordingly, the IL-18 inhibitor, preferably, is an IL-18 binding protein (IL-18BP) which is an IL-18BP, including any functional equivalent or functional part thereof which retains the capability of blocking the proinflammatory activity of IL-18.

In a specific embodiment, said IL-18 inhibitor is an IL-18 binding protein (IL-18BP), particularly human IL-18BP (hIL-18BP), particularly recombinant human IL-18BP (rhIL-18BP), including any functional equivalent or functional part thereof, which retains the capability of blocking the proinflammatory activity of IL-18.

In another specific embodiment, the IL-18BP is selected from isoform a, b, c and d of IL-18BP, particularly isoform a, particularly isoform c, particularly isoform a, b, c or d as shown in SEQ ID NOs 2, and SEQ ID NOs: 3, 4 and 5, but especially isoform a of IL-18BP as shown in SEQ ID NO: 2, or isoform c as shown in SEQ ID NO: 4, including any functional equivalent or functional part thereof, which retains the capability of blocking the proinflammatory activity of IL-18.

Also mixtures of the above isoforms in different combinations may be used in the present invention, but particularly a mixture of isoform a and isoform c, including any functional equivalent or functional part thereof which retains the capability of blocking the proinflammatory activity of IL-18.

Accordingly, a composition comprising the above defined IL-18BP as provided above is also provided for the uses described herein.

Within the present invention, the term “functional” is meant to relate to the equivalent or the part that has still retained the IL-18 blocking activity of the IL-18 inhibitor in the subject's body and is thus capable of blocking the proinflammatory activity of IL-18 and thus to interrupt the immunopathological cascade responsible for VEXAS-related symptoms.

Also comprised within the scope of the present invention is a functional mutein of IL-18BP, a functional fragment, a functional peptide, a functional derivative, a functional fraction, a functional circularly permuted derivative, a functional fused protein comprising IL-18BP, an functional isoform or a functional salt thereof, which retains the capability of blocking the proinflammatory activity of IL-18, preferably which retains the IL-18 blocking activity of the IL-18BP in the subject's body and is thus capable of blocking the proinflammatory activity of free IL-18 and thus to interrupt the immunopathological cascade responsible for VEXAS-related symptoms.

In one embodiment, the invention relates to an IL-18BP, which is a fused protein comprising all or part of an IL-18BP, fused to all or part of an immunoglobulin, preferably to the constant region of an immunoglobulin, and wherein the fused protein is still capable of binding to IL-18, which retains the capability of blocking the proinflammatory activity of free IL-18, and preferably has retained the IL-18 blocking activity of the IL-18BP and is thus capable of blocking the proinflammatory activity of free IL-18 and thus to interrupt the immunopathological cascade responsible for VEXAS-related symptoms. More specifically, the immunoglobulin may be of the IgG1 or IgG2 isotype, for use in a composition according to any one of the embodiments described herein.

In another embodiment the invention relates to an IL-18BP, which is a fused protein comprising all or part of an IL-18BP, fused to all or part of a small molecule, particularly to all or part of a small molecular drug, wherein the fused protein retains the capability of blocking the proinflammatory activity of free IL-18, and preferably is still capable of blocking activity of the IL-18 and is thus capable of blocking the proinflammatory activity of IL-18 and thus to interrupt the immunopathological cascade responsible for VEXAS-related symptoms.

In one embodiment, the present invention provides the IL-18 inhibitor for use as disclosed in any one of the embodiments described herein, wherein the inhibitor is an IL-18 Binding Protein (IL-18BP) which has a sequence identity of 80%, 81%, 82%, 83%, 84%, 85%, 86%, 87%, 88%, 89%, 90%, 91%, 92%, 93%, 94%, 95%, 96%, 97%, 98%, 99% or 100% to the sequence depicted in SEQ ID NO: 2 and SEQ ID NOs: 3, 4 and 5, but particularly to the sequence depicted in SEQ ID NO: 2 and SEQ ID NO: 4 and has retained the IL-18 blocking activity of the IL-18 inhibitor and is thus capable of blocking the proinflammatory activity of IL-18 and thus to interrupt the immunopathological cascade responsible for VEXAS-related symptoms.

In another embodiment of the invention, the IL-18BP or the composition comprising the IL-18BP may in addition comprise N-terminal and/or C-terminal deletion variants of IL-18BP in an amount of up to 40%, particularly up to 30%, particularly up to 20%, particularly up to 15%, particularly up to 10%, particularly up to 7.5%, particularly up to 5%, particularly up to 2.5%, particularly up to 1%, particularly up to 0.5%, particularly up to 0.25%, particularly up to 0.1%, particularly up to 0.05%, particularly up to 0.01%.

In a specific embodiment, said N-terminal and/or C-terminal deletion variants of IL-18BP are present in an amount of up to 40%, particularly in an amount of between 2% and 35%.

In particular, said deletion variants comprise deletions of between 1 and 5 amino acid residues at the C-terminal end of the IL-18BP and/or between 1 and 30 amino acid residues at the N-terminal end of the IL-18BP.

In one embodiment, the invention relates to a composition for use in the treatment of VEXAS or a VEXAS-related symptoms as defined in any one of the embodiments disclosed herein comprising an IL-18 inhibitor as defined in any one of the embodiments disclosed herein and a pharmaceutically acceptable carrier and/or excipient.

In various embodiments of the invention, the IL-18 inhibitor, particularly the IL-18BP of the invention as described herein, including any functional equivalent or functional part thereof, or the composition comprising said IL-18 inhibitor, particularly the IL-18BP of the invention, including any functional equivalent or functional part thereof, is used for the treatment of VEXAS or a VEXAS-related symptoms.

The IL-18 inhibitor, particularly the IL-18BP of the invention, including any functional equivalent or functional part thereof, or the composition comprising said IL-18 inhibitor, particularly the IL-18BP of the invention, including any functional equivalent or functional part thereof, is used for the treatment of VEXAS or a VEXAS-related symptoms.

Treatment of VEXAS or VEXAS-related symptoms according to any one of the preceding embodiments with the IL-18 inhibitor of the invention, particularly the IL-18BP of the invention as defined herein, including any functional equivalent or functional part thereof, or the composition comprising the IL-18 inhibitor of the invention, particularly the IL-18BP of the invention as defined herein, including any functional equivalent or functional part thereof, comprises prevention, halting, alleviation or reversion of symptoms associated with VEXAS.

In another specific embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof, is administered to the subject to be treated at least until the treated subject shows a therapeutic response, in particular alleviation of at least one of the symptoms associated with VEXAS.

In various further embodiment, the invention relates to the IL-18 inhibitor of the invention, particularly the IL-18BP of the invention as defined herein, or the composition comprising the IL-18 inhibitor of the invention, particularly the IL-18BP of the invention as defined herein, for use according to any one of the preceding embodiments, wherein
increased expression of IFN-γ, IL-13 or IL-17A is modified, particularly inhibited, compared to untreated subjects suffering from VEXAS or VEXAS-related symptoms; and/or
binding of free IL-18 by the IL-18BP compensates the IL-18/IL-18BP imbalance by trapping and neutralizing the excess of free IL-18 in tissue and circulation; and/or.
IL-18 binding is restricted or inhibited, particularly binding of free IL-18 to IL-18 receptor (IL-18R), but especially free IL-18 binding to IL-18Rα; and/or
the IL-18BP reduces binding of IL-18 to IL-18 receptor, particularly binding to IL-18Rα by at least 5%, particularly by at least 10%, particularly by at least 15%, particularly by at least 20%, particularly by at least 25%, particularly by at least 30%, particularly by at least 40%, particularly by at least 45%, particularly by at least 50%, particularly by at least 55%, particularly by at least 60%, particularly by at least 65%, particularly by at least 70, particularly by at least 75, particularly by at least 80, particularly by at least 85%, particularly by at least 90%, particularly by at least 95%, particularly by 100%; and/or
the IL-18BP neutralizes free IL-18 by restricting or preventing IL-18 binding to IL-18 receptor (IL-18R), especially free IL-18 binding to IL-18Rα.

In one embodiment, the IL-18 inhibitor is provided for use of any one of the embodiments provided herein, wherein the subject has been diagnosed with an abnormal level of ferritin (FERR), in particular a level of FERR above 400 ng/ml, preferably above 1000 ng/ml, above 1500 ng/ml, above 2000 ng/ml, above 2500 ng/ml, above 3000 ng/ml, or above 3500 ng/ml.

In one embodiment, the invention relates to the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, for use according to any one of the preceding embodiments, wherein the level of free IL-18 in the body fluids and/or body tissues has been determined to be ≥2.7 pg/ml, in particular above about 8 pg/ml.

In a specific embodiment of the invention, the level of free IL-18 in the body fluids and/or body tissues is above the quantification limit (>8 pg/ml).

In another specific embodiment of the invention, the level of free IL-18 in the body fluids and/or body tissues is above the detection limit (>2.7 pg/ml).

In still another specific embodiment, the level of total IL-18 in the body fluids and/or body tissues is abnormal, in particular above about 250 pg/ml, in particular above about 1000 pg/ml, more particular above about 3000 pg/ml.

The determination of free IL-18 in the body fluids and/or body tissues may be accomplished by using an assay for quantifying the level of free IL-18 in the body fluids and/or body tissues in a body sample or in situ, which includes the steps of:
a) bringing a sample of body fluid and/or body tissue or a body part or body area suspected to contain free IL-18 into contact with IL-18BP or an antibody, which specifically binds to free IL-18, but not to IL-18 bound in a complex and functions as the capturing molecule for free IL-18;
b) allowing the IL-18BP or the antibody to bind to free IL-18;
c) detecting the binding of IL-18 to the IL-18BP or the antibody and determining the amount of free IL-18 in the sample or in situ.

In one embodiment, the invention relates to the method according to any one of the preceding embodiments, wherein said sample is selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa, bone-marrow or tissue secretion.

For determining the presence or absence of free IL-18 in a sample according to the method described herein in the various embodiments, any immunoassay format known to those of ordinary skill in the art may be used such as, for example, assay formats which utilize indirect detection methods using secondary reagents for detection. In particular, ELISA's and immunoprecipitation and agglutination assays may be used. A detailed description of these assays is, for example, given in Harlow and Lane, Antibodies: A Laboratory Manual (Cold Spring Harbor Laboratory, New York 1988) 555-612, WO96/13590 to Maertens and Stuyver, Zrein et al. (1998) and WO96/29605.

The sample may be a non-diluted or diluted biological fluid, such as, without being restricted thereto, serum, urine, tear, saliva, bile, sweat, exhalation or expiration, sputum, bronchoalveolar fluid, sebum, cellular, gland, mucosa or tissue secretion, biopsy, homogenized tissue.

For in situ diagnosis, the IL-18BP or the antibody or any active and functional part thereof may be administered to the organism to be diagnosed by methods known in the art such as, for example, intravenous, intranasal, intraperitoneal, intracerebral, intraarterial injection such that a specific binding between the IL-18BP or the antibody with free IL-18 may occur. The antibody/antigen complex may conveniently be detected through a label attached to the antibody or a functional fragment thereof or any other art-known method of detection.

In another aspect of the invention, detection of free IL-18 described herein may be accomplished by an immunoassay procedure. The immunoassay typically includes contacting a test sample with an antibody or the IL-18BP as described herein in the various embodiments that specifically binds to free IL-18 and detecting the presence of the IL-18BP/free IL-18 complex or the antibody/free IL-18 complex in the sample. The immunoassay procedure may be selected from a wide variety of immunoassay procedures known to those skilled in the art such as, for example, competitive or non-competitive enzyme-based immunoassays, enzyme-linked immunosorbent assays (ELISA), radioimmunoassay (RIA), and Western blots, etc. Further, multiplex assays may be used, including arrays, wherein IL-18BP or the antibody are placed on a support, such as a glass bead or plate, and reacted or otherwise contacted with the test sample.

Antibodies used in these assays may be monoclonal or polyclonal, and may be of any type such as IgG, IgM, IgA, IgD and IgE. Antibodies may be produced by immunizing animals such as rats, mice, and rabbits. The antigen used for immunization may be isolated from the samples or synthesized by recombinant protein technology. Methods of producing antibodies and of performing antibody-based assays are well-known to the skilled artisan and are described, for example, more thoroughly in Antibodies: A Laboratory Manual (1988) by Harlow & Lane; Immunoassays: A Practical Approach, Oxford University Press, Gosling, J. P. (ed.) (2001) and/or Current Protocols in Molecular Biology (Ausubel et al.) which is regularly and periodically updated.

Various chemical or biochemical derivatives of the IL-18BP or antibodies or antibody fragments can be produced using known methods. One type of derivative which is diagnostically useful as an immunoconjugate comprising an IL-18BP or an antibody molecule, or an antigen-binding fragment thereof, to which is conjugated a detectable label. However, in many embodiments, the IL-18BP or the antibody is not labeled but in the course of an assay, it becomes indirectly labeled by binding to or being bound by another molecule that is labeled. The invention encompasses molecular complexes comprising an IL-18BP or an antibody molecule and a label.

Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferones, fluoresceins, fluorescein isothiocyanate, rhodamines, dichlorotriazinylamine fluorescein, dansyl chloride, phycoerythrins, Alexa Fluor 647, Alexa Fluor 680, DilC₁₉(3), Rhodamine Red-X, Alexa Fluor 660, Alexa Fluor 546, Texas Red, YOYO-1+DNA, tetramethylrhodamine, Alexa Fluor 594, BODIPY FL, Alexa Fluor 488, Fluorescein, BODIPY TR, BODIPY TMR, carboxy SNARF-1, FM 1-43, Fura-2, Indo-1, Cascade Blue, NBD, DAPI, Alexa Fluor 350, aminomethylcoumarin, Lucifer yellow, Propidium iodide, or dansylamide; an example of a luminescent material includes luminol; examples of bioluminescent materials include green fluorescent proteins, modified green fluorescent proteins, luciferase, luciferin, and aequorin, and examples of suitable radioactive material include ¹²⁵I, ¹³¹I, ³⁵S or ³H.

The immunoassays will typically comprise incubating a sample, such as a biological fluid, a tissue extract, freshly harvested cells, or lysates of cells, in the presence of a detectably labeled IL-18BP or an antibody or peptide fragments thereof, and detecting the bound IL-18BP or antibody by any of a number of techniques well-known in the art. One way of measuring the level of free IL-18 with the IL-18BP or antibody is by enzyme immunoassay (EIA) such as an enzyme-linked immunosorbent assay (ELISA) (Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, FL, 1980). The enzyme, either conjugated to the IL-18BP or the antibody or to a binding partner for the IL-18BP or the antibody, when later exposed to an appropriate substrate, will react with the substrate in such a manner as to produce a chemical moiety which can be detected, for example, by spectrophotometric, or fluorimetric means. In a specific embodiment of the invention, the IL-18BP used in any of the above formats, but particularly in an ELISA format is IL-18BP isoform a, b, c or d, or a functional equivalent or a functional derivate thereof, or a functional fragment thereof, particularly isoform a, particularly isoform c, or a derivate thereof, particularly isoform a, b, c or d as shown in SEQ ID NOs 2, and 3, 4 and 5, but especially the isoform a of IL-18BP as shown in SEQ ID NO: 2 or the isoform c as shown in SEQ ID NO 4.

Also mixtures of the above isoforms may be used in the composition of the invention, but particularly a mixture of isoform a and isoform c.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labelled IL-18BP or antibody. The solid phase support may then be washed with the buffer a second time to remove unbound IL-18BP or antibody. The amount of bound label on solid support may then be detected by conventional means. A well-known example of such a technique is Western blotting.

In various embodiments, the present invention provides compositions comprising labelled IL-18BP or labelled antibodies according to the invention as described herein.

In still another embodiment, the invention relates to a method for treating VEXAS or a VEXAS-related symptom as defined in any one of the preceding embodiments, said method comprising:
a. in a first optional step, quantifying the amount of free IL-18 in the body fluids and/or body tissues of said subject using the method according to the invention and as described herein in the various embodiments;
b. administering to a subject, which suffers from treating VEXAS or a VEXAS-related symptom as defined in any one of the preceding embodiments and has abnormal levels of free IL-18 in the body fluids and/or body tissues, which exceed the level of free IL-18 in body fluids and/or body tissues of a healthy control subject by 5%, 10%, 20%, 30%, 40%, 50%, 60%, 70%, 80%, 90%, 100%, or more than 100%, a therapeutically or prophylactically effective amount of an IL-18 inhibitor or a composition comprising said IL-18 inhibitor as defined in any one of the preceding embodiments, particularly by systemic, intranasal, buccal, oral, transmucosal, intratracheal, intravenous, subcutaneous, intraurinary tract, intravaginal, sublingual, intrabronchial, intrapulmonary, transdermal or intramuscular administration, in particular broncho-pulmonary administration.

In a specific embodiment, the IL-18 inhibitor is the IL-18BP as defined herein in the preceding embodiments.

The IL-18 inhibitor or the pharmaceutical composition comprising the IL-18 inhibitor of the invention and as disclosed herein in the various embodiments is administered to a subject suffering from treating VEXAS or a VEXAS-related symptom as defined herein before in any of the preceding embodiments in suitable dosage forms and units and dosage intervals.

In a specific embodiment, the IL-18 inhibitor, particularly the IL-18BP, of the invention, including any functional equivalent or functional part thereof, as defined herein is formulated as a pharmaceutical composition comprising a sterile solution for injection and further sodium chloride, and/or sodium hydroxide and/or sodium phosphate buffer, particularly in a concentration of between 0.01 M and 0.1 M, particularly between 0.01 M and 0.05 M, but especially of 0.01 M.

In particular, said composition of the invention comprises sodium chloride, sodium hydroxide and a sodium phosphate buffer in a concentration of 0.01 M.

The IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, may be administered to a patient in need thereof in a single dose or dosage unit/day, in multiple doses or dosage units/day, in multiple doses or dosage units/week or in multiple doses or dosage units/month. The single dose or dosage unit may be split into several doses or dosage units and administered to the subject to be treated over several hours or a whole day.

In one embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered in one dose per week, in two doses per week, three doses per week, four doses per week, five doses per week, six doses per week, but particularly seven doses per week.

In another embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered every 24 h to 48 h.

In still another embodiment, the IL-18 inhibitor, particularly the IL-18BP, of the invention, including any functional equivalent or functional part thereof, or to the composition comprising the IL-18 inhibitor, particularly the IL-18BP, of the invention, including any functional equivalent or functional part thereof, is administered in every other day, three or four times per week.

In one embodiment, the composition according to the invention will be administered by subcutaneous (s.c.) injection. In particular, the site of the s.c. injection is alternated, particularly the site of injection is outside of the thighs and the various quadrants of the anterior abdominal wall. The separate injections that constitute a single dosage of the composition of the invention is particularly administered within the same body region but not at the exact same injection site.

In one embodiment, the composition is brought to room temperature, particularly between 18-25° C., before administration.

In a specific embodiment, a single dose of the composition of the invention and particularly the composition for use according to any one of the preceding embodiments comprises between 10 mg and 600 mg IL-18BP.

In particular, the single dose comprises between 10 and 20 mg, between 20 and 40 mg, between 40 and 80 mg, between 80 and 160 mg, between 160 mg and 320 mg or between 320 mg and 600 mg IL-18BP.

In various embodiments of the invention, a single dose comprises between 0.5 mg of IL-18 inhibitor/kg body weight and 10 mg IL-18 inhibitor/kg body weight, particularly between 1 mg IL-18 inhibitor/kg body weight and 8 mg IL-18 inhibitor/kg body weight, particularly between 1.5 mg IL-18 inhibitor/kg body weight and 6 mg IL-18 inhibitor/kg body weight, particularly between 2 mg IL-18 inhibitor/kg body weight and 4 mg IL-18 inhibitor/kg body weight.

In a specific embodiment of the invention, a single dose comprises between 2 mg of IL-18BP/kg body weight and 3 mg IL-18BP/kg body weight, particularly 2 mg of IL-18BP/kg body weight.

In a specific embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof is administered in a dosage of between 0.5 mg IL-18 inhibitor/kg body weight and 5 mg IL-18 inhibitor/kg body weight every 24 or 48 h, particularly, a single dose of 2 mg IL-18 inhibitor/kg body weight is administered every 48 h.

In another specific embodiment, human IL-18BP (rhIL-18 BP), including any functional equivalent or functional part thereof or the composition comprising a recombinant human IL-18BP (rhIL-18 BP), including any functional equivalent or functional part thereof, which retains the capability of blocking the proinflammatory activity of IL-18 is used in the treatment of treating VEXAS or a VEXAS-related symptom as described herein in the various embodiments, wherein the recombinant human IL-18BP (rhIL-18 BP) or the composition comprising the recombinant human IL-18BP (rhIL-18 BP) is administered in a single dose every other day of 2 mg/kg body weight, for example over three weeks.

In still another specific embodiment, a recombinant human IL-18BP (rhIL-18 BP) or a composition comprising the recombinant human IL-18BP (rhIL-18 BP), including any functional equivalent or functional part thereof which retains the capability of blocking the proinflammatory activity of IL-18, is used in the treatment of treating VEXAS or a VEXAS-related symptom in a human patient, which suffers from treating VEXAS or a VEXAS-related symptom and has a level of free IL-18 two to three times above the level of a healthy control subject, wherein said recombinant human IL-18BP (rhIL-18 BP) or the composition comprising the recombinant human IL-18BP (rhIL-18 BP) is administered to said patient in a single dose every other day, three or four times per week, of 2 mg/kg body weight, for example over three weeks.

The compositions of the invention may comprise additional medicinal agents, pharmaceutical agents, carriers, buffers, dispersing agents, diluents, co-therapeutic agents such as anti-inflammatory, bronchodilatory, antihistamine, decongestant, anti-tussive drug substances, antiviral and/or immunosuppressant drugs, and the like, depending on the intended use and application.

In one embodiment of the present invention, the IL-18 inhibitor or the pharmaceutical composition comprising the IL-18 inhibitor of the invention and as disclosed herein in the various embodiments, is administered prophylactically.

In another embodiment of the present invention, the IL-18 inhibitor or the pharmaceutical composition comprising the IL-18 inhibitor of the invention and as disclosed herein in the various embodiments is administered therapeutically.

In one embodiment, the IL-18 inhibitor, particularly the IL-18BP, of the invention, including any functional equivalent or functional part thereof, or the composition comprising the IL-18 inhibitor, particularly the IL-18BP, of the invention, including any functional equivalent or functional part thereof is used in co-medication. Co-medication, either concomitantly or separately, may comprise medicaments known in the treatment or prevention of one or more of the symptoms associated with VEXAS. Symptoms include but are not limited to fever, fatigue, weight loss, lymphadenopathies, arthralgia/arthritis, skin nodular lesions and the like. Symptoms observed in laboratory tests may be anemia, neutropenia, thrombocytopenia, and/or an increase in blood markers such as CRP and ferritin. Myeloid cell vacuolization is also a characteristic sign of VEXAS. Hemophagocytosis is frequently observed in bone marrow that can show, in addition, signs of myelofibrosis. Recurrent skin rash and symmetric polyarthritis are also known as manifestations/symptoms. Inflammation markers are usually elevated, at least during an active phase of the disease, i.e. a phase where symptoms are observed. Accordingly, medicaments known to have an effect on one or more of the symptoms described above are envisaged as co-medication of VEXAS or VEXAS-related symptoms. Such medication may comprise corticosteroid, tocilizumab and/or anakinra, cyclosporine, JAK inhibitor, rituximab, siltiximab, sirolimus, methotrexate, each as sole co-medication or in combination.

In one embodiment of the present invention, the IL-18 inhibitor or the pharmaceutical composition comprising the IL-18 inhibitor of the invention and as disclosed herein in the various embodiments is administered to a subject suffering from treating VEXAS or a VEXAS-related symptoms as defined herein before in any of the preceding embodiments by systemic, intranasal, intraocular, intravitral, eye drops, buccal, oral, transmucosal, intratracheal, intravenous, subcutaneous, intraurinary tract, intrarectal, intravaginal, sublingual, intrabronchial, intrapulmonary, transdermal or intramuscular administration.

The IL-18 inhibitor or the pharmaceutical composition comprising the IL-18 inhibitor of the invention and as disclosed herein in the various embodiments may be provided as a liquid, liquid spray, microspheres, semisolid, gel, or powder for transmucosal administration, e.g. intranasal, buccal, oral transmucosal, intratracheal, intraurinary tract, intravaginal, sublingual, intrabronchial, intrapulmonary and/or transdermal administration. Further, the composition may be in a solid dosage form for buccal, oral transmucosal and/or sublingual administration. Intranasal, buccal, oral intratracheal, intraurinary tract, intravaginal, transmucosal and sublingual administrations lead to the disintegration of the composition as described herein in an oral cavity at body temperature and optionally may adhere to the body tissue of the oral cavity. Additionally, the composition as disclosed herein further may include one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof.

In a specific aspect of the present invention, the IL-18 inhibitor or the pharmaceutical composition comprising the IL-18 inhibitor is formulated as a parenteral, or intravenous solution, suspension, emulsion, as a tablet, pill, bioadhesive patch, drops, sponge, film, lozenge, hard candy, wafer, sphere, lollipop, disc-shaped structure, suppository or spray.

Transmucosal administration is generally rapid because of the rich vascular supply to the mucosa and the lack of a stratum corneum in the epidermis. Such drug transport typically provides a rapid rise in blood concentrations, and similarly avoids the enterohepatic circulation and immediate destruction by gastric acid or partial first-pass effects of gut wall and hepatic metabolism. Drugs typically need to have prolonged exposure to a mucosal surface for significant drug absorption to occur.

The transmucosal routes can also be more effective than the oral route in that these routes can provide for relatively faster absorption and onset of therapeutic action. Further, the transmucosal routes can be preferred for use in treating patients who have difficulty in swallowing tablets, capsules, or other oral solids, or those who have disease-compromised intestinal absorption. Accordingly, there are many advantages to transmucosal administration of the IL-18 inhibitor, particularly the IL-18BP, or a pharmaceutical composition comprising the IL-18 inhibitor, particularly the IL-18BP and a pharmaceutically acceptable carrier and/or excipient.

In either of the intranasal or buccal routes, drug absorption can be delayed or prolonged, or uptake may be almost as rapid as if an intravenous bolus were administered. Because of the high permeability of the rich blood supply, the sublingual route can provide a rapid onset of action.

The intranasal compositions can be administered by any appropriate method according to their form. A composition including microspheres or a powder can be administered using a nasal insufflator device. Examples of these devices are well known to those of skill in the art and include commercial powder systems such as Fisons Lomudal System. An insufflator produces a finely divided cloud of the dry powder or microspheres. The insufflator is preferably provided with a mechanism to ensure administration of a substantially fixed amount of the composition. The powder or microspheres can be used directly with an insufflator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device. Further, the composition can provide an initial rapid release of the active ingredient followed by a sustained release of the active ingredient, for example, by providing more than one type of microsphere or powder. Further, alternative methods suitable for administering a composition to the nasal cavity will be well known by the person of ordinary skill in the art. Any suitable method may be used. For a more detailed description of suitable methods reference is made to EP2112923, EP1635783, EP1648406, EP2112923 (the entire contents of which are incorporated by reference herein).

In one embodiment of the present invention, the IL-18 inhibitor or the pharmaceutical composition comprising the IL-18 inhibitor of the invention and as disclosed herein in the various embodiments may be further administered intranasally, i.e. by inhalation and, thus, may be formulated in a form suitable for intranasal administration, i.e. as an aerosol, dry powder formulation or a liquid preparation.

Examples of suitable pharmaceutical carriers, excipients and/or diluents are well known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulation are aqueous or non-aqueous solutions, suspensions, dry powder formulations, emulsions or oils. Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, fish-liver oil, another marine oil, or a lipid from milk or eggs.

The present invention also relates to transpulmonary administration by inhalation of the IL-18 inhibitor or the pharmaceutical composition comprising the IL-18 inhibitor of the invention and as disclosed herein in the various embodiments is in dry powder, gaseous or volatile formulations into systemic circulation via the respiratory tract. Absorption is virtually as rapid as the formulation can be delivered into the alveoli of the lungs, since the alveolar and vascular epithelial membranes are quite permeable, blood flow is abundant and there is a very large surface for adsorption. For instance, aerosols may be delivered from pressure-packaged, metered-dose inhalers (MDIs).

The IL-18 inhibitor or the pharmaceutical composition comprising the IL-18 inhibitor of the invention and as disclosed herein in the various embodiments would generally be administered in a mixture with a suitable pharmaceutical excipient, diluent or carrier selected with regard to the chosen means of inhalation and standard pharmaceutical practice.

In another embodiment of the invention, the IL-18 inhibitor formulation, particularly the IL-18BP formulation or the formulation of a pharmaceutical composition comprising the IL18 inhibitor, particularly the IL-18BP, is a dry powder, optionally together with at least one particulate pharmaceutically acceptable carrier, which may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially preferred carrier is lactose, for example lactose monohydrate or anhydrous lactose. The dry powder may be contained as unit doses in capsules of, for example, gelatin or plastic, or in blisters (e.g. of aluminium or plastic), for use in a dry powder inhalation device, which may be a single dose or multiple dose device, preferably in dosage units together with the carrier in amounts to bring the total weight of powder per capsule to from 5 mg to 50 mg. Alternatively, the dry powder may be contained in a reservoir in a multi-dose dry powder inhalation (MDDPI) device adapted to deliver.

Any other therapeutically efficacious route of administration can be used, for example absorption through epithelial or endothelial tissues or by gene therapy wherein a DNA molecule encoding the active agent is administered to the patient (e.g. via an expression vector), which causes the active agent to be expressed and secreted in vivo.

The IL-18 inhibitor or the pharmaceutical composition comprising the IL-18 inhibitor of the invention and as disclosed herein in the various embodiments may be used for treatment of VEXAS or a VEXAS-related symptoms described herein in the various embodiments in human and veterinary medicine for treating humans and animals, including avians, non-human primates, dogs, cats, pigs, goats, sheep, cattle, horses, mice, rats and rabbits.

In a specific embodiment, the present invention provides the IL-18 inhibitor or the pharmaceutical composition comprising the IL-18 inhibitor of the invention as disclosed herein in the various embodiments for use in the treatment of VEXAS or a VEXAS-related symptoms as described herein in the various embodiments, wherein the subject is a mammal, in particular the subject is a human.

In another specific embodiment, the pharmaceutical composition of the invention as disclosed herein in the various embodiments is administered in a therapeutically effective amount with a suitable dose of at least a second proinflammatory cytokine inhibitor. In particular said inhibitor is specific for IL-1, IL-6, IL-13, IL-17A, IFN-γ or TNFα.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the biologically active compound of the invention, the compound retains the biological activity.

Efforts have been made in the art to chemically modify the barrier properties of skin to permit the penetration of certain agents, enhance the effectiveness of the agent being delivered, enhance delivery times, reduce the dosages delivered, reduce the side effects from various delivery methods, reduce patient reactions, and so forth.

In this regard, penetration enhancers have been used to increase the permeability of the dermal surface to drugs and are often proton accepting solvents such as dimethyl sulfoxide (DMSO) and dimethylacetamide. Other penetration enhancers that have been studied and reported as effective include 2-pyrrolidine, N,N-diethyl-m-toluamide (Deet), 1-dodecal-azacycloheptane-2-one, N,N-dimethylformamide, N-methyl-2-pyrrolidine, calcium thioglycolate, hexanol, fatty acids and esters, pyrrolidone derivatives, derivatives of 1,3-dioxanes and 1,3-dioxolanes, 1-N-dodecyl-2-pyrrolidone-5-carboxylic acid, 2-pentyl-2-oxo-pyrrolidineacetic acid, 2-dodecyl-2-oxo-1-pyrrolidineacetic acid, 1-azacycloheptan-2-one-2-dodecylacetic acid, and aminoalcohol derivatives, including derivatives of 1,3-dioxanes, among others.

Preparations for transmucosal administration may include sterile aqueous or non-aqueous solutions, suspensions, dry powder formulations and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Transmucosal vehicles may include sodium chloride solution, Ringer's dextrose, dextrose and sodium chloride, lactated Ringer's, or fixed oils. Preservatives and other additives may also be present including, for example, antimicrobials, anti-oxidants, chelating agents, and inert gases and the like. In addition, the pharmaceutical composition of the present invention might comprise proteinaceous carriers, like, e.g., serum albumin or immunoglobulin, preferably of human origin.

The IL-18 inhibitor or the pharmaceutical composition comprising the IL-18 inhibitor of the invention as disclosed herein in the various embodiments may be administered topically to body surfaces and, thus, be formulated in a form suitable for topical administration. Suitable topical formulations include gels, ointments, creams, lotions, drops and the like. For topical administration, the pharmaceutical composition of the invention as disclosed herein in the various embodiments is prepared and applied as a solution, suspension, or emulsion in a physiologically acceptable diluent with or without a pharmaceutical carrier.

The IL-18 inhibitor or the pharmaceutical composition comprising the IL-18 inhibitor of the invention and as disclosed herein in the various embodiments may also be administered as controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

Further examples for suitable formulations are provided in WO 2006/085983, the entire contents of which are incorporated by reference herein. For example, the IL-18 inhibitor or the pharmaceutical composition comprising the IL-18 inhibitor of the invention and as disclosed herein in the various embodiments is of the present invention may be provided as liposomal formulations. The technology for forming liposomal suspensions is well known in the art. The lipid layer employed can be of any conventional composition and can either contain cholesterol or can be cholesterol-free. The liposomes can be reduced in size, as through the use of standard sonication and homogenization techniques. Liposomal formulations containing the pharmaceutical composition of the invention as disclosed herein in the various embodiments can be lyophilized to produce a lyophilizate which can be reconstituted with a pharmaceutically acceptable carrier, such as water, to regenerate a liposomal suspension. The pharmaceutical composition of the invention as disclosed herein in the various embodiments can be administered to the subject at a suitable dose. The dosage regimen will be determined by the attending physician and clinical factors. As is well known in the medical arts, dosages for any one subject depend upon many factors, including the subject's size, body surface area, age, the particular compound to be administered, sex, time and route of administration, general health, and other drugs being administered concurrently.

Furthermore, it is envisaged that the IL-18 inhibitor or the pharmaceutical composition comprising the IL-18 inhibitor of the invention might comprise further biologically active agents, depending on the intended use of the pharmaceutical composition. These further biologically active agents may be e.g. antibodies, antibody fragments, hormones, growth factors, enzymes, binding molecules, cytokines, chemokines, nucleic acid molecules and drugs. In a preferred embodiment, the pharmaceutical composition of the present invention may be co-administered with long-acting beta-adrenoceptor agonist (LABA), long-acting muscarinic antagonists (LAMA), steroids, corticosteroid, glucocorticoid and glucocorticoid agonists phosphodiesterase inhibitors, kinase inhibitors, cytokine and chemokine inhibitors, antibiotics, antagonists or protease inhibitors, anti-viral and/or anti-inflammatory drugs or combinations thereof.

The dosage of the IL-18 inhibitor or the pharmaceutical composition comprising the IL-18 inhibitor of the invention as disclosed herein in the various embodiments will depend on the treating VEXAS-related symptom being treated, the particular composition used, and other clinical factors such as weight, size and condition of the subject, body surface area, the particular compound or composition to be administered, other drugs being administered concurrently, and the route of administration.

The IL-18 inhibitor or the pharmaceutical composition comprising the IL-18 inhibitor of the invention as disclosed herein in the various embodiments may be administered in combination with other biologically active substances and procedures for the treatment of symptoms associated VEXAS. The other biologically active substances may be part of the same composition already comprising the composition according to the invention, in form of a mixture, wherein the composition of the invention and the other biologically active substance are intermixed in or with the same pharmaceutically acceptable solvent and/or carrier or may be provided separately as part of a separate compositions, which may be offered separately or together in form of a kit of parts.

The IL-18 inhibitor or the pharmaceutical composition comprising the IL-18 inhibitor of the invention as disclosed herein in the various embodiments may be administered concomitantly with the other biologically active substance or substances, intermittently or sequentially. For example, the composition according to the invention may be administered simultaneously with a first additional biologically active substance or sequentially after or before administration of said composition. If an application scheme is chosen where more than one additional biologically active substance are administered and at least one composition according to the invention, the compounds or substances may be partially administered simultaneously, partially sequentially in various combinations.

It is thus another object of the present invention to provide for mixtures of the IL-18 inhibitor or the pharmaceutical composition comprising the IL-18 inhibitor of the invention as disclosed herein in the various embodiments, optionally comprising, one or more further biologically active substances in a therapeutically or prophylactically effective amount, as well as to methods of using such a mixture for prevention and/or therapeutic treatment.

The other biologically active substance or compound may exert its biological effect by the same or a similar mechanism as the composition according to the invention or by an unrelated mechanism of action or by a multiplicity of related and/or unrelated mechanisms of action.

Generally, the other biologically active compound may include antibodies raised against and binding to IFN-γ, IL-17A, IL-13, IL-1beta, IL-6, IL-2, IL-4, IL-12, TNF-alpha. In particular, the mixture according to the invention may comprise IL-18BP (IL-18BP) or a pharmaceutical composition comprising IL-18BP (IL-18BP) and a pharmaceutically acceptable carrier and/or excipient according to the invention and as described herein.

Suitable dosages of the pharmaceutical composition of the invention as disclosed herein in the various embodiments will vary depending upon the condition, age and species of the subject, and can be readily determined by those skilled in the art. The total daily dosages of the employed in both veterinary and human medicine will suitably be in the range of 0.1 to 10 mg per kilogram.

Further, functional derivatives of the IL-18 inhibitor, particularly the IL-18BP, may be conjugated to polymers in order to improve the properties of the protein, such as the stability, half-life, bioavailability, tolerance by the human body, or immunogenicity. To achieve this goal, IL-18BP may be linked e.g. to Polyethlyenglycol (PEG). PEGylation may be carried out by known methods, described in WO 92/13095, for example.

Therefore, in another embodiment of the present invention, IL-18BP is PEGylated.

In still another embodiment of the invention, IL-18BP is a fused protein comprising all or part of an IL-18BP, which is fused to all or part of an immunoglobulin, preferably to the constant region (Fc) of an immunoglobulin, and wherein the fused protein is still capable of binding to IL-18. More specifically, the immunoglobulin may be of the IgG1 or IgG2 isotype.

In a further embodiment of the invention, the IL-18BP is PEGylated, fused to all or part of an immunoglobulin, preferably to the constant region (Fc) of an immunoglobulin, and wherein the fused protein is still capable of binding to IL-18. More specifically, the immunoglobulin may be of the IgG1 or IgG2 isotype.

The person skilled in the art will understand that the resulting fusion protein retains the biological activity of IL-18BP, in particular the binding to IL-18. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the IL-18BP sequence and the immunoglobulin sequence. The resulting fusion protein has improved properties, such as an extended residence time in body fluids (half-life), increased specific activity, increased expression level, or the purification of the fusion protein is facilitated.

Preferably, it is fused to heavy chain regions, like the CH2 and CH3 domains of human IgG1, for example. The generation of specific fusion proteins comprising IL-18BP and a portion of an immunoglobulin are described in example 11 of WP99/09063, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero or homomultimeric.

### Definitions

The technical terms and expressions used within the scope of this application are generally to be given the meaning commonly applied to them in the pertinent art if not otherwise indicated herein below.

As used in this specification and the appended embodiments, the singular forms “a”, “an”, and “the” include plural referents unless the context clearly dictates otherwise. Thus, for example, reference to “a compound” includes one or more compounds.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effects attributed to the disease. The term “treatment” as used herein covers any treatment of a disease in a subject and includes: (a) preventing a disease, i.e. related to an undesired immune response from occurring in a subject which may be predisposed to the disease; (b) inhibiting the disease, i.e. arresting its development; or (c) relieving the disease, i.e. causing regression of the disease (d) reversing the disease symptoms, i.e. leading to recovery of damaged tissue.

The expression “IL-18 Binding Protein (IL-18BP)” as used herein includes the full-length protein, a mutein, fragment, peptide, functional derivative, functional fragment, fraction, circularly permuted derivative, fused protein comprising IL-18BP, isoform or a salt thereof.

The term “free IL-18” as used herein means monomeric, soluble and non-complexed interleukin-18 protein.

The term “functional” and “active” are used herein synonymously and refers to a modified IL-18 inhibitor, particularly a modified IL-18BP, or to a part or fragment of an IL-18 inhibitor, particularly a part or fragment of IL-18BP, or to an equivalent of an IL-18 inhibitor, particularly an equivalent of IL-18BP, which still have/retains the same or essentially the same biological, pharmacological and therapeutic properties as the unmodified or full length IL-18 inhibitor, particularly the unmodified or full length IL-18BP, and can thus be used within the present invention for the treatment of the diseases and disorders as disclosed herein the same way as the unmodified or full length IL-18 inhibitor, particularly as the unmodified or full length IL-18BP. In particular, by “functional” is meant that the modified, partial or equivalent IL-18 inhibitor, particularly the modified, partial or equivalent IL-18BP, has still retained the IL-18 blocking activity of the unmodified or full length IL-18 inhibitor, particularly the unmodified or full length IL-18BP, in treating VEXAS or a VEXAS-related symptom and is thus capable of blocking the proinflammatory activity of IL-18 and thus to interrupt the immunopathological cascade responsible treating VEXAS or a VEXAS-related symptom.

In various embodiments of the invention, the term “IL-18BP” refers to human IL-18BP, particularly to recombinant human IL-18BP, particularly to isoform a, b, c or d of IL-18BP, particularly isoform a, particularly isoform c, particularly isoform a, b, c or d as shown in SEQ ID NOs 2, and SEQ ID NOs: 3, 4 and 5, but especially the isoform a of IL-18BP as shown in SEQ ID NO: 2 or the isoform c as shown in SEQ ID NO 4.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as [kappa] and [lambda] light chains. Heavy chains are classified as [micro], [Delta], [gamma], [alpha], or [epsilon], and define the antibody's isotype as IgM, IgD, IgG, IgA, and IgE, respectively. Within light and heavy chains, the variable and constant regions are joined by a “J” region of about 122 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR.2, FR3, CDR3 and FR4. The assignment of amino acids to each domain is in accordance with the definitions of Kabat Sequences of Proteins of Immunological Interest. The Kabat Complementarity Determining Regions are based on sequence variability and are the most commonly used (National Institutes of Health, Bethesda, Md. (1987 and 1991), or Chothia & Lesk J. Mol. Biol, 196:901-917 (1987)).

Chothia refers instead to the location of the structural loops (Chothia and Lesk J. Mol. Biol. 196:901-917 (1987), Chothia et al. Nature 342:878-883 (1989)).

An alternative system for the assignment of amino acids to each domain is the IMGT system (http://www.imgt.org/IMGTScientificChart/Nomenclature/IMGT-FRCDRdefinition.html). The terms “antibody” or “antibodies” as used herein are art recognized term and are understood to refer to molecules or active fragments of molecules that bind to known antigens, particularly to immunoglobulin molecules and to immunologically active portions of immunoglobulin molecules, i.e. molecules that contain a binding site that immunospecifically binds an antigen. The immunoglobulin according to the invention can be of any type (IgG, IgM, IgD, IgE, IgA and IgY) or class (IgG1, IgG2, IgG3, IgG4, IgA1 and IgA2) or subclasses of immunoglobulin molecule.

The term “Antibody” refers for the purpose of the present invention to an intact immunoglobulin or to an antigen-binding portion thereof that competes with the intact antibody for specific binding. In particular, “Antibodies” are intended within the scope of the present invention to include monoclonal, polyclonal, chimeric, single chain, bispecific or bi-effective, simianized, human and humanized antibodies.

Examples of Antigen-binding portions include, inter alia, Fab, Fab′, F(ab′)2, scFv, dAb and Fv fragments, including the products of a Fab immunoglobulin expression library and epitope-binding fragments of any of the antibodies and fragments mentioned above. Further examples of Antigen-binding portions include complementarity determining region (CDR) fragments, diabodies and polypeptides that contain at least a portion of an immunoglobulin that is sufficient to confer specific antigen binding to the polypeptide.

Such active fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. ct al. J. Nucl. Med. 23:1011-1019 (1982); Rousseaux et al. Methods Enzymology, 121:663-69, Academic Press, 1986.

A “patient” or “subject” for the purposes of the present invention is used interchangeably and meant to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

The expressions “pharmaceutical composition” and “therapeutical composition” are used herein interchangeably in the widest sense. They are meant to refer, for the purposes of the present invention, to a therapeutically effective amount of the active ingredient, i.e. the IL-18BP and, optionally, a pharmaceutically acceptable carrier or diluent.

It embraces compositions that are suitable for the curative treatment, the control, the amelioration, an improvement of the condition or the prevention of a disease or disorder in a human being or a non-human animal. Thus, it embraces pharmaceutical compositions for the use in the area of human or veterinary medicine. Such a “therapeutic composition” is characterized in that it embraces at least one IL-18BP compound or a physiologically acceptable salt thereof, and optionally a carrier or excipient whereby the salt and the carrier and excipient are tolerated by the target organism that is treated therewith.

A “therapeutically effective amount” refers to that amount which provides a therapeutic effect for a given condition and administration regimen. In particular, “therapeutically effective amount” means an amount that is effective to prevent, reverse, alleviate or ameliorate symptoms of the disease or prolong the survival of the subject being treated, which may be a human or non-human animal. Determination of a therapeutically effective amount is within the skill of the person skilled in the art. In particular, in the present case a “therapeutically or prophylactically effective amount” refers to the amount of protein or peptide, mutein, functional derivative, fraction, circularly permuted derivative, fused protein, isoform or a salt thereof, and compound or pharmaceutical composition which, when administered to a human or animal, leads to a therapeutic or prophylactic effect in said human or animal. The effective amount is readily determined by one of skill in the art following routine procedures. The therapeutically effective amount or dosage of a compound according to this invention can vary within wide limits and may be determined in a manner known in the relevant art. The dosage can vary within wide limits and will, of course, have to be adjusted to the individual requirements in each particular case.

The term “transmucosal” administration refers to various administration routes wherein the compound is absorbed by the mucosa of any part of the body. Transmucosal administration comprises, but is not limited to, i.e. intranasal, buccal, oral transmucosal, intratracheal, intraurinary tract, intrarectal, intravaginal, sublingual, intrabronchial, intrapulmonary and transdermal administration.

The definition “pharmaceutically acceptable” is meant to encompass any carrier, excipient, diluent or vehicle, which does not interfere with effectiveness of the biological activity of the active ingredient and that is not toxic to the host to which it is administered.

The term “fused protein” refers to a polypeptide comprising an IL-18BP, or a viral IL-18BP, or a mutein or fragment thereof, fused with another protein, which, e. g., has an extended residence time in body fluids. An IL-18BP or a viral IL-18BP may thus be fused to another protein, polypeptide or the like, e. g., an immunoglobulin or a fragment thereof.

These isoforms, muteins, fused proteins or functional derivatives retain the biological activity of IL-18BP, in particular the binding to IL-18, and preferably have essentially at least an activity similar to IL-18BP. Ideally, such proteins have a biological activity which is even increased in comparison to unmodified IL-18BP. Preferred active fractions have an activity which is better than the activity of IL-18BP, or which have further advantages, like a better stability or a lower toxicity or immunogenicity, or they are easier to produce in large quantities, or easier to purify.

The term “interleukin-18 binding protein” (IL-18BP) comprises also functional equivalents of the IL-18BP, including mutein, functional derivative, fraction, biologically active peptide, circularly permuted derivative, fused protein, isoform and a salt thereof.

In particular, the functional equivalent of the IL-18BP may have a sequence identity of 80%, 85%, 90%, 91%, 92%, 93%, 94%, 95%, 96%, 97%, 98%, 99% to the sequence depicted in SEQ ID NO: 2 and which retains the capability of blocking the proinflammatory activity of IL-18, preferably which retains the IL-18 blocking activity of the IL-18BP in virus-induced infectious processes in the lung and is thus capable of blocking the proinflammatory activity of IL-18 and thus to interrupt the immunopathological cascade responsible for irreversible lung damage.

As used herein the term “muteins” refers to analogs of an IL-18BP, or analogs of a viral IL-18BP, in which one or more of the amino acid residues of a natural IL-18BP or viral IL-18BP are replaced by different amino acid residues, or are deleted, or one or more amino acid residues are added to the natural sequence of an IL-18BP, or a viral IL-18BP, without changing considerably the activity of the resulting products as compared with the wild type IL-18BP or viral IL-18BP. These muteins are prepared by known synthesis and/or by site-directed mutagenesis techniques, high throughput mutagenesis, DNA shuffling, protein evolution techniques, or any other known technique suitable therefore.

Any such mutein preferably has a sequence of amino acids sufficiently duplicative of that of an IL-18BP, or sufficiently duplicative of a viral IL-18BP, such as to have substantially similar activity to IL-18BP. One activity of IL-18BP is its capability of binding IL-18. As long as the mutein has substantial binding activity to IL-18, it can be used in the purification of IL-18, such as by means of affinity chromatography, and thus can be considered to have substantially similar activity to IL-18BP. Thus, it can be determined whether any given mutein has substantially the same activity as IL-18BP by means of routine experimentation comprising subjecting such a mutein, e. g. to a simple sandwich competition assay to determine whether or not it binds to an appropriately labelled IL-18, such as radioimmunoassay or ELISA assay.

Muteins of IL-18BP polypeptides or muteins of viral IL-18BPs, which can be used in accordance with the present invention, or nucleic acid coding therefore, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of IL-18BP polypeptides or proteins or viral IL-18BPs, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e. g. under thirty, and preferably under ten, and do not remove or displace amino acids which are critical to a functional conformation, e. g., cysteine residues. Proteins and muteins produced by such deletions and/or insertions come within the purview of the present invention.

“Functional derivatives” as used herein cover derivatives of IL-18BPs or a viral IL-18BP, and their muteins and fused proteins, which may be prepared from the functional groups which occur as side chains on the residues or the N- or C-terminal groups, by means known in the art, and are included in the invention as long as they remain pharmaceutically acceptable, i. e. they do-not destroy the activity of the protein which is substantially similar to the activity of IL-18BP, or viral IL-18BPs, and do not confer toxic properties on compositions containing it.

These derivatives may, for example, include polyethylene glycol side-chains, which may mask antigen sites and extend the residence of an IL-18BP or a viral IL-18BP in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e. g. alkanol or carbocyclic aroyl groups) or O-acyl derivatives of free hydroxyl groups (for example that of seryl or threonyl residues) formed with acyl moieties.

As “functional fragment” of an IL-18BP, or a viral IL-18BP, mutein and fused protein, the present invention covers any fragment or precursors of the polypeptide chain of the IL-18BP protein molecule alone or together with associated molecules or residues linked thereto, e. g., sugar or phosphate residues, or aggregates of the protein molecule or the sugar residues by themselves, provided said fraction has substantially similar activity to IL-18BP.

The term “salts” herein refers to both salts of carboxyl groups and to acid addition salts of amino groups of the IL-18BP molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of IL-18BP, e. g. the ability to bind IL-18.

“Isoforms” of IL-18BP are proteins capable of binding IL-18 or fragment thereof, which may be produced by alternative splicing.

The term “circularly permuted derivatives” as used herein refers to a linear molecule in which the termini have been joined together, either directly or through a linker, to produce a circular molecule, and then the circular molecule is opened at another location to produce a new linear molecule with termini different from the termini in the original molecule. Circular permutations include those molecules whose structure is equivalent to a molecule that has been circularized and then opened. Thus, a circularly permuted molecule may be synthesized de novo as a linear molecule and never go through a circularization and opening step. The preparation of circularly permutated derivatives is described in WO 95/27732.

The expression “abnormal levels of free IL-18” refers to increased or decreased levels of free IL-18 compared to the values detected in body fluids and/or body tissues of a healthy control subject. In particular, these abnormal levels mean increased and detectable values of free IL-18. In particular, said abnormal level of free IL-18 in the body fluids and/or body tissues are levels of free IL-18, which are above the quantification limit, in particular >12 pg/ml and/or above the detection limit, in particular >4 pg/ml of free IL-18, particularly levels of free IL-18, which exceed the level in body fluids and/or body tissues of a healthy control subject by 5%, 10%, 20%, 30%, 40%, 50%, 60%, 70%, 80%, 90%, 100%, or more than 100%. In certain embodiments of the invention the reference or control value is the normal, non-pathologic base value for free IL-18 determined in the patient to be treated. The skilled person is aware that detection and/or quantification limit of free IL-18 may depend on the employed method for determining free IL-18. Therefore, what is possible to be detected may change with the development of improved and/or alternative methods.

The expression “abnormal ratio of free IL-18/IL-18BP” refers to an increased ratio of IL-18 to IL-18BP compared to values found in body fluids and/or body tissues of a healthy control subject. In particular, said abnormal ratio of free IL-18 to IL-18BP in the body fluids and/or body tissues exceeds the ratio in body fluids and/or body tissues of a healthy control subject by 1%, 2.5%, 5%, 10%, 20%, 30%, 40%, 50%, 60%, 70%, 80%, 90%, 100%, or more than 100%. In certain embodiments of the invention the reference or control value is the normal, non-pathologic base value for free IL-18 determined in the patient to be treated.

The expressions “gene silencing” and “post transcriptional gene silencing” mean the suppressive regulation of gene expression by mechanisms others than genetic modification. The silencing occurs by mRNA neutralization on the post transcriptional level, wherein mRNA translation is prevented to form an active gene product, which is in most cases a protein.

The term “predisposition” means the increased susceptibility of a subject for developing a specific disease. In the present case a subject is classified as predisposed if for instance elevated IL-18 level appear in the lung, serum, sputum, broncho-alveolar lavage fluid (BALF) or circulation.

“Alveolar macrophages” are a subtype of macrophages found in the pulmonary alveolus. They often contain granules of exogenous material that they have picked up from the respiratory surfaces. Such black granules are especially common in people, which are long-time exposed to fine dust, fine particles, e.g. like smoker or long-term city dwellers.

A “Th2 cytokine response” mediated by IL-4, IL-5, IL-6, IL-8, IL-10, IL-13, and/or IL-17A, particularly IL-4 and/or IL-8 and/or IL-17A, whereas a “Th1 cytokine response” is mediated by interferon-gamma (IFN-γ), IL-2, and tumor necrosis factor-alpha (TNF-α).

The expression “IL-18/IL-18BP imbalance” relates to the dysregulation of mutual interaction of IL-18 and IL-18BP, which finally leads to an elevated level of unbound IL-18.

A “disease” is a state of health of a subject, particularly a human, wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a symptom of the disease or disorder, the frequency with which such a symptom is experienced by a subject, or both, are reduced.

The terms “dysregulated” or “dysregulation,” as used herein, refer to an impairment in a biological process which in turn may lead to deleterious physiological sequela, or abnormal expression of a gene, nucleic acid, protein, peptide, or other biological molecule. In the case where expression of a gene, nucleic acid, protein, peptide, or other biological molecule is dysregulated, the gene, nucleic acid, protein, peptide, or other biological molecule is expressed, processed, or maintained at levels that are outside what is considered the normal range for that of that gene, nucleic acid, protein, peptide, or other biological molecule as determined by a skilled artisan. Dysregulation of a gene, nucleic acid, protein, peptide, or other biological molecule in a mammal may be determined by measuring the level of a gene, nucleic acid, protein, peptide, or other biological molecule in the mammal and comparing the level measured in that mammal to level measured in a matched population known not to be experiencing dysregulation of that gene, nucleic acid, protein, peptide, or other biological molecule dysregulated. Alternatively, the level may be compared to one measured in the same individual at a different time.

As used herein “endogenous” refers to any material from or produced inside an organism, cell, tissue or system. The term “exogenous” refers to any material introduced from or produced outside an organism, cell, tissue or system.

The terms “inhibit”, “neutralize” or “block” as used herein, have to be understood as synonyms which mean reducing a molecule, a reaction, an interaction, a gene expression, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

### BRIEF DESCRIPTION OF THE SEQUENCES AND FIGURES

SEQ ID NO 1:
13-amino acid Linker Sequence of hIL-18BP:
Glu-Phe-Gly-Ala-Gly-Leu-Val-
Leu-Gly-Gly-GIn-Phe-Met
SEQ ID NO 2:
(SEQ ID NO 2)
TPVSQTTTAA TASVRSTKDP CPSQPPVFPA
AKQCPALEVT WPEVEVPLNG TLSLSCVACS
RFPNFSILYW LGNGSFIEHL PGRLWEGSTS
RERGSTGTQL CKALVLEQLT PALHSTNFSC
VLVDPEQVVQ RHVVLAQLWA GLRATLPPTQ
EALPSSHSSP QQQG
SEQ ID NO 3:
Amino Acid Sequence of IL-18 Binding
Protein (hIL-18BP), isoform b
(SEQ ID NO 3)
TPVSQTTTAA TASVRSTKDP CPSQPPVFPA
AKQCPALEVT WPEVEVPL SWAEGNLAPH
PRSPALQPQQ STAAGLRLST GPAAAQP
SEQ ID NO: 4:
Amino Acid Sequence of IL-18 Binding
Protein (hIL-18BP), isoform c
(SEQ ID NO 4)
TPVSQTTTAA TASVRSTKDP CPSQPPVFPA
AKQCPALEVT WPEVEVPLNG TLSLSCVACS
RFPNFSILYW LGNGSFIEHL PGRLWEGSTS
RERGSTGTQL CKALVLEQLT PALHSTNFSC
VLVDPEQVVQ RHVVLAQLWV RSPRRGLQEQ
EELCFHMWGK GGLCQSSL
SEQ ID NO: 5:
Amino Acid Sequence of IL-18
Binding Protein (hIL-18BP), isoform d
(SEQ ID NO 5)
TPVSQTTTAA TASVRSTKDP CPSQPPVFPA
AKQCPALEVT WPEVEVPLNG TLSLSCVACS
RFPNFSILYW LGNGSFIEHL PGRLWEGSTS
RERGSTGWAE GNLAPHPRSP ALQPQQSTAA
GRLSTGPAAA QP

FIG. 1: Distribution of total IL-18 levels within the VEXAS cohort.

FIG. 2: Distribution of free IL-18 levels within the “VEXAS cohort-64”.

FIG. 3: Correlation between total IL-18 and free IL-18 levels. The analysis was performed on the “VEXAS cohort-64”, excluding three outliers.

FIG. 4: Enrichment of free IL-18 in patients with high levels of total IL-18. The analysis was performed on the “VEXAS cohort-64”.

FIG. 5: Correlation between total IL-18 and IL-18BP levels. The analysis was performed on the whole VEXAS cohort, excluding one outlier.

### EXAMPLES

A broad genotype-driven study revealed that the genetic cause of a rare and often fatal inflammatory disease, the VEXAS (vacuoles, E1 enzyme, X-linked, autoinflammatory, somatic) syndrome, was an acquired somatic mutation in UBA1 on the X chromosome. This gene encodes the major E1 enzyme involved in the activation of ubiquitin, a small regulatory protein which attaches to substrate proteins in a process named ubiquitylation. Mutation of the methionine 41 of UBA1 results in decreased ubiquitylation, particularly in haematopoietic stem cells with subsequent activation of the innate immune system. Patients affected with the VEXAS syndrome develop inflammatory and hematologic symptoms.

Although initially thought to be rare since its first description in October 2020, it is being increasingly diagnosed on retrospective genetic testing of patient cohorts identified by clinical features including series from France, the Netherlands, and the USA, as well as individual case reports. The VEXAS syndrome is almost exclusively described in males, with most cases diagnosed in mid to late adult life. There has been one female with VEXAS syndrome reported who had a single X-chromosome.

The VEXAS syndrome is clinically heterogeneous, but has been recently described as the association between severe autoinflammatory manifestations mostly involving the skin and bone marrow and myeloid dysplasia. While patients typically poorly respond to treatment, some clinical improvement has been observed with high dose immunosuppressive drugs (methotrexate or corticosteroids), pro-inflammatory cytokine targeting agents (tocilizumab or adalimumab) and signalling inhibitors (cyclosporin or JAK inhibitors). Nevertheless, most of these treatments revealed to be only transiently effective on inflammation, with no real improvement in cytopenia, thus the VEXAS syndrome presents high unmet medical needs.

Patients diagnosed with the VEXAS syndrome present severe autoinflammatory manifestations, with recurrent fever and elevated inflammatory markers. Likewise in other autoinflammatory diseases, these patients experience an uncontrolled immune activation in the form of a Cytokine Release Syndrome (CRS). The management of the CRS is currently dominated by the use of corticosteroids, tocilizumab and/or anakinra, the latter drugs blocking the IL-6 and the IL-1 stimulatory pathway, respectively. Nevertheless, these therapeutic approaches have been reported to show only limited efficacy. One of the working hypotheses to decipher the molecular bases of CRS is that it results from the induction of two distinct cytokine stimulatory pathways, the IL-1B/IL-6 one and the IL-18/IFN-γ one.

IL-18 is a pro-inflammatory cytokine belonging to the IL-1 family, first identified for its IFN-γ-inducing properties. By binding to IL-18, IL-18 binding protein (IL-18BP) neutralizes it and acts as a key regulator of the immune responses. Under healthy conditions, all IL-18 is bound by IL-18BP and there are no or little amounts of free active IL-18. In patients suffering from a systemic inflammatory disorder, both IL-18 and IL-18BP levels are drastically increased, but due to the higher elevation of IL-18, IL-18BP is not able to efficiently neutralize the active free form of IL-18, which leads to a pathological presence of free IL-18. Free IL-18 which is not inactivated by IL-18 BP, induces the immune system constantly and can lead to severe autoinflammatory situations and CRS with a potentially fatal outcome.

High levels of IL-18 have been measured in the serum of a fraction of patients diagnosed with the VEXAS syndrome (unpublished results) and these findings suggest that this rare and deadly autoinflammatory disorder may involve the IL-18 pathway.

This study aims at bringing evidence of the involvement of the IL-18 pathway in the VEXAS syndrome and of the pathogenically appearance of free active IL-18 in the serum of patients affected with this autoinflammatory disorder. This could support a rationale of administrating exogenous rhIL-18BP, such as Tadekinig alfa, currently in development by AB2 Bio for an efficacious neutralization of the uncontrolled IL-18 activity, helping the resolution of the hyperinflammation.

Serum samples from a cohort of 73 VEXAS syndrome patients were monitored in this study. Free IL-18 and IL-18BP measurements were conducted, using an AB2 Bio proprietary and commercially available (DY119 from R&D) assays, respectively. Both assays are based on a sandwich ELISA technique and detection of the analytes is performed using a colorimetric reagent.

Serum samples were tested in duplicates and the analyte concentrations were extrapolated from an internal calibration curve. These concentrations were qualified when the coefficient of variation between duplicates was acceptable (CV<40%). Since 64 out of 73 samples generated qualified values for the measurement of free IL-18, the analysis of free IL-18 data was restricted to this set of samples and the corresponding patient sub-group was referred to as “VEXAS cohort-64”.

The statistical analyses were performed using the built-in regression function of the Excel software (Microsoft Office 2019). The generated regression curves and correlation coefficients (R²) were reported onto the scatterplot graphs. The correlation coefficient indicates the correlation between a pair of variables. Correlation coefficients are between −1 and 1; a correlation coefficient around 0 indicates no linear relationship between two variables (a cloud of points with no obvious structure linking the two variables); the closer the value approaches 1, the stronger the positive linear association. Similarly, the closer the value approaches −1, the stronger the negative linear association (when one value increases, the other one tends to decrease).

### Results

### High Total IL-18 Levels in the VEXAS Cohort

The serum samples of the VEXAS cohort present total IL-18 levels, ranging from 145 pg/ml to 9,339 pg/mL, with a median value of 1,719 pg/ml and a mean value of 2,247 pg/mL. The concentrations of total IL-18 in the 73 serum samples of the VEXAS cohort are reported in Table 3.

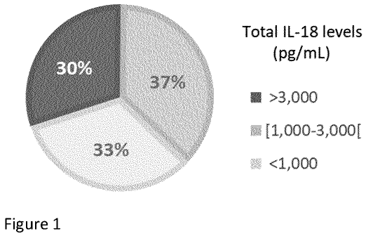

Twenty-two patients out of 73 present very high levels of total IL-18 levels (>3,000 μg/mL), 49 having high levels (>1,000 μg/mL) (FIG. 1).

TABLE 1
Distribution of total IL-18 levels within the VEXAS cohort

| Total IL-18 (pg/mL) | Number | Fraction |
|---|---|---|
| ≥3,000 | 22 | 30 |
| [1,000-3,000[ | 27 | 37 |
| <1,000 | 24 | 33 |

### Detectable Levels of Free IL-18 in the “VEXAS Cohort-64”

The raw data of the free IL-18 measurements in serum of the VEXAS cohort are reported in the Table 4. Among the 73 measured samples, 64 provided qualified free IL-18 concentration, i.e. presenting acceptable variation between duplicates (CV<40%) and constitute the “VEXAS cohort-64” set of samples.

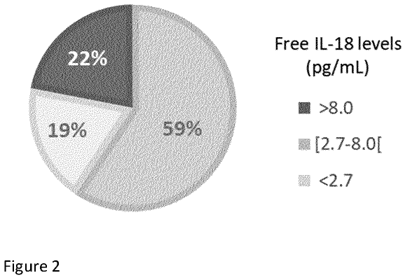

The serum samples from the “VEXAS cohort-64” present free IL-18 levels, ranging from 0.76 pg/mL to 21.03 pg/mL, with a median value of 4.40 pg/mL and a mean value of 5.80 pg/mL. Fourteen patients out of 64 present quantifiable levels of free IL-18 (>8.0 pg/mL), while 52 show detectable levels (>2.7 pg/mL) (FIG. 2).

TABLE 2
Distribution of free IL-18 levels within the “VEXAS cohort-64”

| Free IL-18 (pg/mL) | Number | Fraction |
|---|---|---|
| ≥8.0 | 14 | 22 |
| [2.7-8.0[ | 38 | 59 |
| <2.7 | 12 | 19 |

### Correlation Between Free IL-18 and Total IL-18 Levels

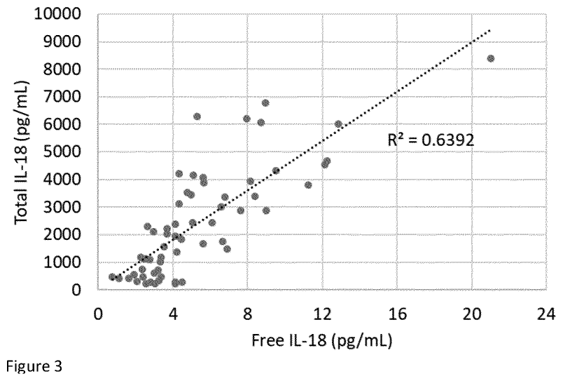

The patients presenting the highest total IL-18 levels are also the ones presenting high free IL-18 levels. The evaluation of the correlation between these two parameters was performed on the “VEXAS cohort-64” sub-group of patients. The calculated correlation coefficient (R²=0.6392) indicates a linear positive correlation between total IL-18 and free IL-18 parameters (FIG. 3).

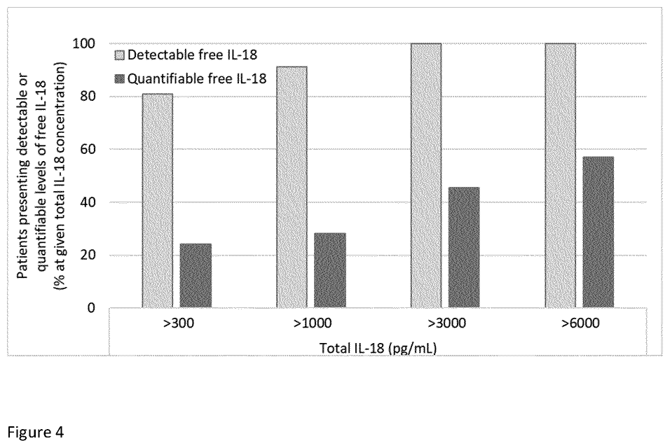

There is an enrichment of free IL-18 in patients with high total IL-18 levels. While quantifiable levels of free IL-18 can be found in 24% of the patients with above normal total IL-18 levels (>300 g/mL), this fraction reaches 57% in patients with very high total IL-18 levels (>6,000 μg/mL). Similarly, the fraction of patients presenting detectable levels of free IL-18 increases from 81% to 100% from patients with normal to very high total IL-18 levels (FIG. 4).

### No Concomitant Increase of IL-18BP and Total IL-18 Levels

The serum samples from the VEXAS cohort present IL-18BP levels, ranging from 20,077 pg/mL to 303,422 pg/mL, with a median value of 50,018 pg/mL and a mean value of 60,401 pg/mL. The raw data of the IL-18BP measurements are reported in the Table 5.

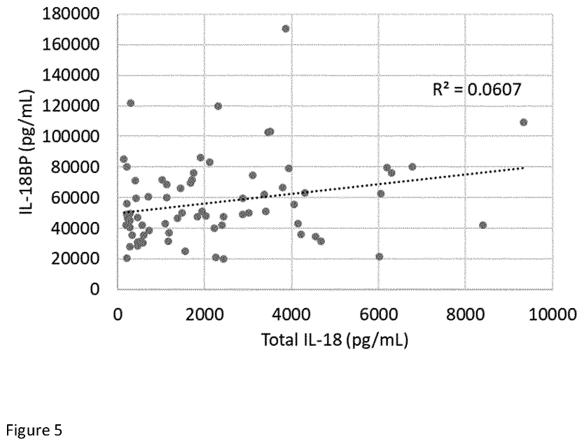

The calculated correlation coefficient (R²=0.0607) indicates no correlation between total IL-18 and IL-18BP parameters (FIG. 5).

Monitoring of total IL-18 in the serum of the VEXAS syndrome cohort reveals that all measured values except seven are above the normal concentration (>260 μg/mL). A third of the patients (22 out of 73) presents very high levels of total IL-18 levels (>3,000 μg/mL), while two thirds (49 out of 73) have high levels (>1,000 μg/mL). This demonstrates that the autoinflammation affecting these patients involves induction of the IL-18 pathway.

Nearly one fourth of these patients (14 out of 64) presents quantifiable levels of free IL-18 (>8.0 pg/mL), while 81% (52 out of 64) show detectable levels (>2.7 pg/mL). Moreover, the increase in free IL-18 levels correlates with the one of the total IL-18. Since free IL-18 is the active pathological form of IL-18, its detection in the serum of the majority of these patients strongly suggests that the autoinflammation they experienced is triggered, at least in part, by the excessive elevation of IL-18, and by the resulting appearance of free IL-18 species.

This inflammation is accompanied by the elevation of IL-18BP levels, as all patients present above the normal levels of IL-18BP (>2,000 μg/mL). However, the IL-18BP levels do not correlate with the ones of total IL-18, providing the molecular basis for the detection of free IL-18 species: the increase in the amounts of IL-18BP relative to the ones of IL-18 is not sufficient to bind and keep IL-18 in an inactive status, leading to the appearance of pro-inflammatory free IL-18 species.

These findings indicate a strong implication of the IL-18 pathway in patients affected by the VEXAS syndrome and the lack of association between total IL-18 and IL-18BP elevations supports the therapeutic approach consisting of an exogenous supply of IL-18BP to help neutralizing the increased IL-18 activity, thus facilitating the control of the hyperinflammation.

TABLE 3
Total IL-18 concentrations

| Patient ID | Total IL-18 (pg/mL) |
|---|---|
| UPN01 | 9339 |
| UPN02 | 211 |
| UPN03 | 429 |
| UPN04 | 145 |
| UPN05 | 210 |
| UPN06 | 8400 |
| UPN07 | 1088 |
| UPN08 | 1454 |
| UPN09 | 1719 |
| UPN10 | 1170 |
| UPN11 | 6195 |
| UPN12 | 2878 |
| UPN13 | 3796 |
| UPN14 | 284 |
| UPN15 | 1740 |
| UPN16 | 3873 |
| UPN17 | 2428 |
| UPN18 | 559 |
| UPN19 | 2313 |
| UPN20 | 3011 |
| UPN21 | 2117 |
| UPN22 | 285 |
| UPN23 | 1023 |
| UPN24 | 3402 |
| UPN25 | 6057 |
| UPN26 | 1950 |
| UPN27 | 714 |
| UPN28 | 212 |
| UPN29 | 466 |
| UPN30 | 1188 |
| UPN31 | 197 |
| UPN32 | 4209 |
| UPN33 | 3110 |
| UPN34 | 1678 |
| UPN35 | 281 |
| UPN36 | 6291 |
| UPN37 | 590 |
| UPN38 | 1127 |
| UPN39 | 2393 |
| UPN40 | 1475 |
| UPN41 | 213 |
| UPN42 | 4146 |
| UPN43 | 2220 |
| UPN44 | 3455 |
| UPN45 | 4684 |
| UPN46 | 3517 |
| UPN47 | 229 |
| UPN48 | 3928 |
| UPN49 | 1841 |
| UPN50 | 2872 |
| UPN51 | 606 |
| UPN52 | 466 |
| UPN53 | 336 |
| UPN54 | 2033 |
| UPN55 | 6011 |
| UPN56 | 4063 |
| UPN57 | 2266 |
| UPN58 | 3373 |
| UPN59 | 1124 |
| UPN60 | 730 |
| UPN61 | 305 |
| UPN62 | 4307 |
| UPN63 | 1382 |
| UPN64 | 1904 |
| UPN65 | 4545 |
| UPN66 | 456 |
| UPN67 | 1027 |
| UPN68 | 414 |
| UPN69 | 1551 |
| UPN70 | 280 |
| UPN71 | 6782 |
| UPN72 | 528 |
| UPN73 | 2432 |

TABLE 4
Free IL-18 raw data

| | Free IL-18 | | | | | |
|---|---|---|---|---|---|---|
| | Value 1 | Value 2 | Mean | CV | Repeat | Conc. |
| Patient ID | (pg/mL) | (pg/mL) | (pg/mL) | (%) | (pg/mL) | (pg/mL) |
| UPN01 | 6.21 | 6.21 | 6.21 | 0.00 | | 6.21 |
| UPN02 | 4.14 | 4.14 | 4.14 | 0.00 | | 4.14 |
| UPN03 | <min | 1.11 | 1.11 | | | 1.11 |
| UPN04 | 5.29 | 1.58 | 3.43 | 76.49 | | 3.43 |
| UPN05 | 6.90 | 3.68 | 5.29 | 43.12 | | 5.29 |
| UPN06 | 20.57 | 21.48 | 21.03 | 3.06 | | 21.03 |
| UPN07 | 2.05 | 3.44 | 2.75 | 36.00 | | 2.75 |
| UPN08 | 3.44 | 0.87 | 2.16 | 84.51 | | 2.16 |
| UPN09 | 1.81 | 0.63 | 1.22 | 68.57 | | 1.22 |
| UPN10 | 2.05 | 2.51 | 2.28 | 14.49 | | 2.28 |
| UPN11 | 7.82 | 8.05 | 7.93 | 2.04 | | 7.93 |
| UPN12 | 12.16 | 6.44 | 9.30 | 43.45 | 9.00 | 9.00 |
| UPN13 | 14.21 | 8.28 | 11.24 | 37.31 | | 11.24 |
| UPN14 | 4.83 | 4.14 | 4.49 | 10.92 | | 4.49 |
| UPN15 | 5.75 | 7.59 | 6.67 | 19.46 | | 6.67 |
| UPN16 | 6.44 | 4.83 | 5.64 | 20.20 | | 5.64 |
| UPN17 | 5.06 | 5.06 | 5.06 | 0.00 | | 5.06 |
| UPN18 | 1.81 | 2.05 | 1.93 | 8.59 | | 1.93 |
| UPN19 | 2.05 | 3.21 | 2.63 | 31.34 | | 2.63 |
| UPN20 | 7.13 | 5.98 | 6.56 | 12.38 | | 6.56 |
| UPN21 | 2.75 | 3.21 | 2.98 | 11.04 | | 2.98 |
| UPN22 | 3.68 | 4.60 | 4.14 | 15.80 | | 4.14 |
| UPN23 | 2.98 | 5.52 | 4.25 | 42.30 | | 4.25 |
| UPN24 | 9.42 | 7.36 | 8.39 | 17.36 | | 8.39 |
| UPN25 | 8.73 | 8.73 | 8.73 | 0.00 | | 8.73 |
| UPN26 | 3.91 | 4.37 | 4.14 | 7.90 | | 4.14 |
| UPN27 | 2.75 | 3.68 | 3.21 | 20.45 | | 3.21 |
| UPN28 | 10.56 | 3.21 | 6.89 | 75.46 | 3.05 | 3.05 |
| UPN29 | 2.51 | 2.28 | 2.40 | 6.89 | | 2.40 |
| UPN30 | 4.12 | 2.57 | 3.35 | 32.78 | | 3.35 |
| UPN31 | 0.17 | 0.43 | 0.30 | 63.40 | | 0.30 |
| UPN32 | 4.34 | 4.34 | 4.34 | 0.00 | | 4.34 |
| UPN33 | 5.20 | 3.46 | 4.33 | 28.37 | | 4.33 |
| UPN34 | 5.42 | 5.84 | 5.63 | 5.35 | | 5.63 |
| UPN35 | 0.94 | 0.43 | 0.68 | 51.86 | | 0.68 |
| UPN36 | 5.20 | 5.42 | 5.31 | 2.85 | | 5.31 |
| UPN37 | 6.27 | 3.02 | 4.64 | 49.44 | | 4.64 |
| UPN38 | 3.02 | 2.11 | 2.57 | 24.91 | | 2.57 |
| UPN39 | 3.90 | 4.34 | 4.12 | 7.48 | | 4.12 |
| UPN40 | 6.05 | 7.73 | 6.89 | 17.23 | | 6.89 |
| UPN41 | 7.73 | 2.34 | 5.04 | 75.65 | | 5.04 |
| UPN42 | 5.42 | 4.77 | 5.09 | 8.94 | | 5.09 |
| UPN43 | 3.46 | 3.90 | 3.68 | 8.44 | | 3.68 |
| UPN44 | 4.12 | 5.84 | 4.98 | 24.42 | | 4.98 |
| UPN45 | 12.24 | 12.24 | 12.24 | 0.00 | | 12.24 |
| UPN46 | 4.77 | 4.77 | 4.77 | 0.00 | | 4.77 |
| UPN47 | 2.34 | 2.80 | 2.57 | 12.44 | | 2.57 |
| UPN48 | 7.94 | 8.36 | 8.15 | 3.60 | | 8.15 |
| UPN49 | 4.77 | 4.12 | 4.45 | 10.34 | | 4.45 |
| UPN50 | 7.52 | 7.73 | 7.63 | 1.93 | | 7.63 |
| UPN51 | 3.02 | 3.02 | 3.02 | 0.00 | | 3.02 |
| UPN52 | 2.80 | 3.90 | 3.35 | 23.38 | | 3.35 |
| UPN53 | 3.46 | 3.02 | 3.24 | 9.69 | | 3.24 |
| UPN54 | 3.68 | 3.68 | 3.68 | 0.00 | | 3.68 |
| UPN55 | 12.44 | 13.24 | 12.84 | 4.43 | | 12.84 |
| UPN56 | 5.42 | 5.84 | 5.63 | 5.35 | | 5.63 |
| UPN57 | 15.64 | 12.64 | 14.14 | 15.01 | | 14.14 |
| UPN58 | 6.69 | 6.90 | 6.79 | 2.19 | | 6.79 |
| UPN59 | 2.26 | 2.62 | 2.44 | 10.27 | | 2.44 |
| UPN60 | 2.26 | 2.44 | 2.35 | 5.33 | | 2.35 |
| UPN61 | 2.09 | 2.09 | 2.09 | 0.00 | | 2.09 |
| UPN62 | 10.31 | 8.69 | 9.50 | 12.06 | | 9.50 |
| UPN63 | 4.40 | 4.04 | 4.22 | 5.97 | | 4.22 |
| UPN64 | 15.38 | 16.66 | 16.02 | 5.63 | | 16.02 |
| UPN65 | 12.12 | 12.12 | 12.12 | 0.00 | | 12.12 |
| UPN66 | 0.85 | 0.67 | 0.76 | 16.37 | | 0.76 |
| UPN67 | 3.68 | 2.97 | 3.33 | 15.10 | | 3.33 |
| UPN68 | 1.73 | 1.56 | 1.64 | 7.61 | | 1.64 |
| UPN69 | 3.15 | 3.86 | 3.51 | 14.34 | | 3.51 |
| UPN70 | 2.79 | 2.79 | 2.79 | 0.00 | | 2.79 |
| UPN71 | 8.87 | 9.05 | 8.96 | 1.42 | | 8.96 |
| UPN72 | 16.66 | 17.39 | 17.03 | 3.03 | | 17.03 |
| UPN73 | 5.64 | 6.54 | 6.09 | 10.38 | | 6.09 |

Free IL-18 levels were measured in duplicates from 73 patient samples and their means were qualified if the coefficient of variation (CV) was below 40%. Mean values obtained with a CV >40% (in bold) are shown in italics and excluded from the final set of 64 qualified values.
A repeat testing was performed for two samples presenting significant free IL-18 levels (>6 pg/mL), but with unacceptable CV.
Free IL-18 levels are not highlighted (<2.7 pg/mL), highlighted in light grey (between 2.7 and 8 pg/mL) or in dark grey (>8 pg/mL).

TABLE 5
IL-18BP raw data

IL-18BP

| | Value 1 | Value 2 | Mean | CV | Conc. |
|---|---|---|---|---|---|
| Patient ID | (pg/mL) | (pg/mL) | (pg/mL) | (%) | (pg/mL) |
| UPN01 | 109919 | 108852 | 109385 | 0.7 | 109385 |
| UPN02 | 55489 | 56520 | 56005 | 1.3 | 56005 |
| UPN03 | 58518 | 61110 | 59814 | 3.1 | 59814 |
| UPN04 | 87007 | 83436 | 85221 | 3.0 | 85221 |
| UPN05 | 45543 | 52767 | 49155 | 10.4 | 49155 |
| UPN06 | 41439 | 42560 | 42000 | 1.9 | 42000 |
| UPN07 | 42988 | 43085 | 43036 | 0.2 | 43036 |
| UPN08 | 69614 | 62629 | 66122 | 7.5 | 66122 |
| UPN09 | 73571 | 70039 | 71805 | 3.5 | 71805 |
| UPN10 | 32268 | 30728 | 31498 | 3.5 | 31498 |
| UPN11 | 79162 | 80528 | 79845 | 1.2 | 79845 |
| UPN12 | 49262 | 49302 | 49282 | 0.1 | 49282 |
| UPN13 | 66668 | 67056 | 66862 | 0.4 | 66862 |
| UPN14 | 40140 | 41238 | 40689 | 1.9 | 40689 |
| UPN15 | 78263 | 73830 | 76046 | 4.1 | 76046 |
| UPN16 | 171395 | 169834 | 170615 | 0.6 | 170615 |
| UPN17 | 46737 | 48076 | 47407 | 2.0 | 47407 |
| UPN18 | 42270 | 41902 | 42086 | 0.6 | 42086 |
| UPN19 | 124951 | 114303 | 119627 | 6.3 | 119627 |
| UPN20 | 51169 | 48866 | 50018 | 3.3 | 50018 |
| UPN21 | 80550 | 85655 | 83103 | 4.3 | 83103 |
| UPN22 | 53049 | 46188 | 49618 | 9.8 | 49618 |
| UPN23 | 73808 | 69423 | 71616 | 4.3 | 71616 |
| UPN24 | 50294 | 52388 | 51341 | 2.9 | 51341 |
| UPN25 | 63127 | 62360 | 62743 | 0.9 | 62743 |
| UPN26 | 52188 | 49599 | 50893 | 3.6 | 50893 |
| UPN27 | 61017 | 59845 | 60431 | 1.4 | 60431 |
| UPN28 | 78438 | 81856 | 80147 | 3.0 | 80147 |
| UPN29 | 30035 | 32174 | 31104 | 4.9 | 31104 |
| UPN30 | 38957 | 34952 | 36954 | 7.7 | 36954 |
| UPN31 | 43224 | 40945 | 42084 | 3.8 | 42084 |
| UPN32 | 36281 | 36119 | 36200 | 0.3 | 36200 |
| UPN33 | 73543 | 75422 | 74482 | 1.8 | 74482 |
| UPN34 | 71325 | 68408 | 69867 | 3.0 | 69867 |
| UPN35 | 44724 | 45599 | 45162 | 1.4 | 45162 |
| UPN36 | 78090 | 73922 | 76006 | 3.9 | 76006 |
| UPN37 | 30278 | 30508 | 30393 | 0.5 | 30393 |
| UPN38 | 69789 | 67916 | 68852 | 1.9 | 68852 |
| UPN39 | 40232 | 43945 | 42089 | 6.2 | 42089 |
| UPN40 | 49374 | 50874 | 50124 | 2.1 | 50124 |
| UPN41 | 19946 | 21565 | 20755 | 5.5 | 20755 |
| UPN42 | 42468 | 43612 | 43040 | 1.9 | 43040 |
| UPN43 | 42082 | 38557 | 40320 | 6.2 | 40320 |
| UPN44 | 102945 | 102368 | 102656 | 0.4 | 102656 |
| UPN45 | 30985 | 32491 | 31738 | 3.4 | 31738 |
| UPN46 | 102176 | 104467 | 103322 | 1.6 | 103322 |
| UPN47 | 47978 | 45208 | 46593 | 4.2 | 46593 |
| UPN48 | 74935 | 82952 | 78944 | 7.2 | 78944 |
| UPN49 | 46159 | 49128 | 47643 | 4.4 | 47643 |
| UPN50 | 61070 | 58105 | 59588 | 3.5 | 59588 |
| UPN51 | 36155 | 34648 | 35401 | 3.0 | 35401 |
| UPN52 | 29679 | 27905 | 28792 | 4.4 | 28792 |
| UPN53 | 35562 | 35795 | 35679 | 0.5 | 35679 |
| UPN54 | 48373 | 47733 | 48053 | 0.9 | 48053 |
| UPN55 | 21513 | 21202 | 21358 | 1.0 | 21358 |
| UPN56 | 55062 | 55858 | 55460 | 1.0 | 55460 |
| UPN57 | 21409 | 20978 | 21194 | 1.4 | 21194 |
| UPN58 | 63933 | 60415 | 62174 | 4.0 | 62174 |
| UPN59 | 60852 | 59250 | 60051 | 1.9 | 60051 |
| UPN60 | 38897 | 38303 | 38600 | 1.1 | 38600 |
| UPN61 | 128033 | 115982 | 122008 | 7.0 | 122008 |
| UPN62 | 62256 | 63605 | 62930 | 1.5 | 62930 |
| UPN63 | 46070 | 46737 | 46404 | 1.0 | 46404 |
| UPN64 | 88300 | 84487 | 86394 | 3.1 | 86394 |
| UPN65 | 34666 | 34098 | 34382 | 1.2 | 34382 |
| UPN66 | 48155 | 46090 | 47122 | 3.1 | 47122 |
| UPN67 | 302853 | 303992 | 303422 | 0.3 | 303422 |
| UPN68 | 70103 | 72410 | 71257 | 2.3 | 71257 |
| UPN69 | 25326 | 24327 | 24827 | 2.8 | 24827 |
| UPN70 | 28876 | 26865 | 27871 | 5.1 | 27871 |
| UPN71 | 79668 | 80550 | 80109 | 0.8 | 80109 |
| UPN72 | 31779 | 31065 | 31422 | 1.6 | 31422 |
| UPN73 | 19013 | 21141 | 20077 | 7.5 | 20077 |

IL-18BP levels were measured in duplicates from 73 patient samples and their means were qualified if the coefficient of variation (CV) was below 40%.

1. A method of treating a VEXAS syndrome or symptoms associated with a VEXAS syndrome in a subject, the method comprising administering to the subject an IL-18 inhibitor.

2. The method of claim 1, wherein symptoms associated with VEXAS syndrome are characterized by autoinflammatory manifestations, in particular severe autoinflammatory manifestations, and/or hyperinflammation, in particular hyperinflammation as characterized by known inflammation markers, in particular elevated known inflammation markers, such as CRP.

3. The method of claim 1, wherein the subject has one or more mutations in the UBA1 gene, in particular at gene locus p11.3 on the X-chromosome.

4. The method of claim 3, wherein the one or more mutations result in an alternative, in particular shorter, isoform of the UBA-1 gene product.

5. The method of claim 4, wherein the one or more mutations comprise a M41T, a M41V, or a M41L substitution.

6. The method of claim 1, wherein treatment is achieved and/or supported by blocking the proinflammatory activity of IL-18.

7. The method of claim 1, wherein the IL-18 inhibitor is:
(a) an IL-18 binding protein (IL-18BP);
(b) a human IL-18BP (hIL-18BP); or
(c) a recombinant human IL-18BP (rhIL-18BP),
including any functional equivalent or functional part thereof which retains the capability of blocking the proinflammatory activity of IL-18.

8-9. (canceled)

10. The method of claim 7, wherein said human IL-18BP is selected from isoform a, b, c and d of human IL-18BP, particularly isoform a as in SEQ ID NO: 2, isoform b as in SEQ ID NO: 3, isoform c as in SEQ ID NO: 4 or isoform d as in SEQ ID NO: 5, including any functional equivalent or functional part of isoforms a, b, c and/or d which retains the capability of blocking the proinflammatory activity of IL-18.

11. The method of claim 7, wherein said human IL-18BP is an IL-18BP as shown in SEQ ID NO: 2, including any functional equivalent or functional part thereof which retains the capability of blocking the proinflammatory activity of IL-18.

12. The method of claim 11 wherein
(a) the functional equivalent has a sequence identity of 80%, 85%, 90%, 91%, 92%, 93%, 94%, 95%, 96%, 97%, 98%, 99% to the sequence depicted in SEQ ID NO: 2 and retains the capability of blocking the proinflammatory activity of IL-18; or
(b) the functional equivalent or functional part thereof includes a mutein of IL-18BP, a fragment, a peptide, a functional derivative, a functional fragment, a fraction, a circularly permuted derivative, a fused protein comprising IL-18BP, an isoform or a salt thereof which retains the capability of blocking the proinflammatory activity of IL-18.

13. (canceled)

14. The method of claim 10, wherein the IL-18BP comprises in addition to the IL-18 binding protein (IL-18BP), N-terminal and/or C-terminal deletion variants of IL-18BP in an amount of up to 0.01%, 0.05%, 0.1%, 0.25%, 0.5%, 1%, 2.5%, 5%, 7.5%, 10%, 15%, 20%, 30%, or 40%.

15. The method of claim 14, wherein said deletion variants comprise deletions of between 1 and 5 amino acid residues at the C-terminal end of the IL-18BP and/or between 1 and 30 amino acid residues at the N-terminal end of the IL-18BP.

16. (canceled)

17. The method of claim 1, wherein body fluids and/or body tissues of the subject to be treated have been quantified to have abnormal levels of free IL-18, which exceed the level of free IL-18 in body fluids and/or body tissues of a healthy control subject by 5%, 10%, 20%, 30%, 40%, 50%, 60%, 70%, 80%, 90%, 100%, or more than 100%, using an assay capable of detecting free IL-18 in body fluids and/or body tissues.

18. The method of claim 17, wherein the subject to be treated has:
(a) a level of free IL-18 above about 2.7 pg/ml, in particular above about 8 pg/ml or wherein the body fluids and/or body tissues of the subject to be treated have been quantified to have a level of free IL-18 above about 2.7 pg/ml, in particular above about 8 pg/ml;
(b) an abnormal, elevated level of total IL-18; or
(c) a level of ferritin above 400 ng/ml, preferably above 1000 ng/ml or wherein the body fluids and/or body tissues of the subject to be treated have been quantified to have a level of ferritin above 400 ng/ml, preferably above 1000 ng/ml.

19-20. (canceled)

21. The method of claim 17, wherein the level of total IL-18 is above about 250 pg/ml, in particular above about 1000 pg/ml, more particular above about 3000 pg/ml or wherein the body fluids and/or body tissues of the subject to be treated have been quantified to have a level of total IL-18 above about 250 pg/ml, in particular above about 1000 pg/ml, more particular above about 3000 pg/ml.

22. (canceled)

23. The method of claim 17, wherein quantifying the level of free IL-18 in the body fluids and/or body tissues comprises the following steps:
a) bringing a sample of body fluid and/or body tissue suspected to contain free IL-18 into contact with an IL-18 inhibitor as the capturing molecule for free IL-18;
b) allowing the IL-18 inhibitor to bind free IL-18;
c) detecting the binding of the IL-18 inhibitor and determining the amount of free IL-18 in the sample.

24. The method of claim 17, wherein the body fluids and/or body tissues are selected from the group consisting of broncho-alveolar lavage fluid (BALF) circulation fluids, secretion fluids, biopsy, and homogenized tissue, particularly serum, urine, tear, saliva, bile, sweat, exhalation or expiration, sputum, bronchoalveolar fluid, sebum, cellular, gland, mucosa, bone-marrow or tissue secretion.

25. The method of claim 1, wherein the IL-18 inhibitor is administered in a pharmaceutical composition with a pharmaceutically acceptable carrier and/or excipient.

26. The method of claim 1, wherein said IL-18 inhibitor or a pharmaceutical composition comprising said IL-18 inhibitor is administered to a subject in need thereof:
(a) in a single dose/day, in multiple doses/day, in multiple doses/week or in multiple doses/month;
(b) in one dose per week, two doses per week, three doses per week, four doses per week, five doses per week, six doses per week, or seven doses per week;
(c) every 24 hours to 48 hours; or
(d) a single dose every other day.

27-29. (canceled)

30. The method of claim 26, wherein a single dose comprises between 0.5 mg of IL-18 inhibitor/kg body weight and 10 mg IL-18 inhibitor/kg body weight.

31. The method of claim 26, wherein a single dose of between 0.5 mg IL-18 inhibitor/kg body weight and 5 mg IL-18 inhibitor/kg body weight is administered every 24 or 48 h.

32. The method of claim 1, wherein the subject to be treated is:
(a) a mammal; and/or
(b) a human.

33. (canceled)

34. The method of claim 7, wherein the IL-18 inhibitor is the recombinant human IL-18BP (rhIL-18 BP), including any functional equivalent or functional part thereof which retains the capability of blocking the proinflammatory activity of IL-18, wherein the subject is human and has a detectable level of free IL-18, wherein said recombinant human IL-18BP (rhIL-18 BP) or a pharmaceutical composition comprising the recombinant human IL-18BP (rhIL-18 BP) is administered to said human in a single dose of 2 mg/kg body weight every 48 h.

35. The method of claim 34, wherein the human suffering from VEXAS syndrome or symptoms associated therewith shows uncontrolled systemic inflammatory reactions and has abnormal, elevated levels of total IL-18.

36. The method of claim 34, wherein the human subject has:
(a) a level of free IL-18 in the body fluids or body tissues above 2.7 pg/mL;
(b) a level of total IL-18 above about 250 pg/ml, above about 1000 pg/ml, or above about 3000 pg/ml or the body fluids and/or body tissues of the human subject have been quantified to have a level of total IL-18 above about 250 pg/ml above about 1000 pg/ml, or above about 3000 pg/ml; and/or
(c) a level of ferritin above 400 ng/ml or above 1000 ng/ml.

37. (canceled)

38. (canceled)

39. (canceled)


SEQUENCE LISTING (5 sequences)

SEQ ID NO: 1 - protein, 13 aa, synthetic construct
  REGION 1..13: note = 13-amino acid Linker Sequence of hIL-18BP
EFGAGLVLGGQFM

SEQ ID NO: 2 - protein, 164 aa, synthetic construct
  REGION 1..164: note = Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 3 - protein, 85 aa, synthetic construct
  REGION 1..85: note = Amino Acid Sequence of IL-18 Binding Protein (hIL-18BP)
TPVSQTTTAATASVRSTKDPCPSQPPVFPAAKQCPALEVTWPEVEVPLSWAEGNLAPHPRSPALQPQQSTAAGLRLSTGPAAAQP

SEQ ID NO: 4 - protein, 168 aa, synthetic construct
  REGION 1..168: note = Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWVRSPRRGLQEQEELCFHMWGKGGLCQSSL

SEQ ID NO: 5 - protein, 132 aa, synthetic construct
  REGION 1..132: note = Amino Acid Sequence of IL-18 Binding Protein (hIL-18BP)
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGWAEGNLAPHPRSPALQPQQSTAAGRLSTGPAAAQP